(19) United States
(12) Patent Application Publication
(10) Pub. No.: US 2013/0288275 A1
(43) Pub. Date: Oct. 31, 2013

(54) COMPOSITIONS AND METHODS FOR DETECTING ACETYLATED SUMO PROTEINS

(71) Applicant: Maria Laura Avantaggiati, Kensington, MD (US)

(72) Inventor: Maria Laura Avantaggiati, Kensington, MD (US)

(73) Assignee: Georgetown University, Washington, DC (US)

(21) Appl. No.: 13/696,422

(22) PCT Filed: May 6, 2011
(86) PCT No.: PCT/US11/35578

§ 371 Date: Jun. 14, 2013

Related U.S. Application Data
(60) Provisional application No. 61/332,002, filed on May 6, 2010.

(51) Int. Cl. G01N 33/574

(52) U.S. Cl. CPC G01N 33/574
USPC 435/7.92; 435/7.1; 530/389.7; 530/387.3; 530/388.85

(57) ABSTRACT

The invention is directed to methods of using antibodies or antibody fragments that specifically bind to an acetylation domain of a small ubiquitin-like modifier protein (SUMO protein) when the acetylation domain is at least partially acetylated. The invention is also directed towards antibodies or antibody fragments that specifically bind to an acetylation domain of a small ubiquitin-like modifier protein (SUMO protein) when the acetylation domain is at least partially acetylated.

### CROSS-REFERENCE TO RELATED APPLICATIONS

This application claims priority to U.S. Provisional Application No. 61/332,002, filed 6 May 2010, which is incorporated by reference.

### STATEMENT REGARDING FEDERALLY SPONSORED RESEARCH OR DEVELOPMENT

Part of the work performed during development of this invention utilized U.S. Government funds under National Institutes of Health Grant Nos. RO1 CA102476 and R21CA123234. The U.S. Government has certain rights in this invention.

### BACKGROUND OF THE INVENTION

1. Field of the Invention

The invention is directed towards methods for determining the susceptibility of cells within a tissue for the induction of cell death, with the methods comprising determining the state of acetylation of an acetylation domain of a small ubiquitin-like modifier protein (SUMO protein) in a sample of the cells obtained from the tissue. At least a partial acetylation of the acetylated domain of the SUMO protein indicates that the cells within the tissue are susceptible to induction of cell death.

2. Background of the Invention

Proteins belonging to the SUMO family (small ubiquitin-like modifiers) members belong to a group of post-translational modifiers that share similar three-dimensional fold with ubiquitin and are essential for transcription, DNA repair, cell survival and for genomic stability. These important processes are unregulated in virtually all tumors. There are four currently known members of the SUMO family, SUMO-1-4, each of which has been shown to play independent effects in regulating the activity of various intracellular proteins. SUMO-1 exists in the cells as a free, non conjugated form, as well as covalently conjugated to a variety of substrates.

Sumoylation is the process by which SUMO proteins attach to target proteins to modify the function of the target protein. Targets of sumoylation include but are not limited to transcription factors, histones and chromatin remodeling enzymes, such as acetylases and deacetylases. Compared to chemical post-translational modifications, SUMO proteins offer a larger surface that can function as a recruitment platform for regulating the interaction of their targets with other proteins in a more complex fashion. Further, given that attachment of SUMO to proteins occurs on lysine residues, which are also recipients of other regulatory modifications, such as acetylation, methylation and ubiquitylation, it is possible that an antagonistic relationship exists between sumoylation and other post-translational events. It is currently envisioned that through a combination of these mechanisms, SUMO moieties convey transcriptional activation or repression, and affect the sub-cellular localization, the stability, and protein-protein interactions of their targets. For example, it is well known that SUMO-1 can either stimulate or inhibit cell proliferation, but the molecular mechanisms by which SUMO-1 achieves such versatility of effects is not clear.

One of the most important factors that is targeted by sumoylation is the transcription regulator and tumor suppressor p53 protein. Particularly, the ability of p53 to induce apoptosis is thought to be a fundamental way by which cells undergo cell death in response to DNA damage and to treatment with radiation and chemo-therapy. In fact, there is evidence that tumor cells lacking a functional p53 are resistant to radiation or chemotherapy. A large percentage of tumors, however, possess an intact and functional p53 gene, such that p53 is active.

The inventors have discovered that SUMO-1 is acetylated at lysine residues conserved in all SUMO family members. This “acetylation domain” in SUMO proteins comprises a stretch of five lysine residues, all of which can be acetylated both in vitro and in vivo. The inventors have also discovered that the acetylation state of the SUMO acetylation domain (SAD, Sumo Acetylation Domain) is important for regulation of the activity of sumoylated proteins, such as p53. In addition, SAD is nested within the surface of SUMO that binds to SUMO-Interacting-Motifs, (SIM), contained in several SUMO target proteins that, in turn, regulate a variety of biological processes ranging from transcription, DNA repair, chromatin, and cell survival.

### SUMMARY OF THE INVENTION

The invention is directed towards methods for determining the susceptibility of cells within a tissue for the induction of cell death, with the methods comprising determining the state of acetylation of an acetylation domain of a small ubiquitin-like modifier protein (SUMO protein) in a sample of the cells obtained from the tissue. At least a partial acetylation of the acetylated domain of the SUMO protein indicates that the cells within the tissue are susceptible to induction of cell death.

The invention is also directed towards antibodies or antibody fragments that specifically bind to an acetylation domain of a small ubiquitin-like modifier protein (SUMO protein) when the acetylation domain is at least partially acetylated.

### BRIEF DESCRIPTION OF THE DRAWINGS

FIG. 1. Similarity of the N-terminus of SUMO-1 with acetylated domains of transcription factors. A. The p53 C-terminal region from amino acids 300 to 384 was blasted against SUMO-1 sequences by using the LALIGN or ALIGN program at the Gene-stream server (xylian.igh.cnrs.fr/). Known sites of acetylation in p53 are highlighted in red. Arrows, at the top or the bottom, indicate the relevant lysines in p53 or SUMO1 that display identity. B-C. Alignment of the N-terminal region of SUMO-1 with known acetylated domains of YY1 and GATA-1. In both A and B, amino acids that show identity are denoted by double dots, while those with similarity are denoted by single dots. Known acetylated residues in YY1 and GATA-1 are indicated at the bottom of each panel. D. The region of SUMO-1 comprised between amino acids 28 to 58 was analyzed for the presence of potential acetylation motifs, and compared to p53. The Prediction of Nₑ-Acetylation of Internal Lysines (PAIL) program was employed for such analysis available on the world wide web at “bioinformatics.lcd-ustc.org/pail/”. Searches were performed in high stringency conditions and by using a high threshold to yield higher specificity performance (threshold of 0.5). Sites of acetylation in p53 and SUMO and their relative scores, as identified by PAIL, are highlighted in red.

FIG. 2. Identification of an acetylated domain of SUMO-1 conserved among SUMO family members. A. Purified SUMO1 was subjected to an in vitro acetylation reaction in the absence (lane 2) or presence (lane 3) of CBP, and of 14C-Acetyl-Coenzyme A. Lane 1 contain CBP in the absence of SUMO-1. Reactions were assembled in 30 μl volume, and developed with autoradiography. 1/20 of the reaction mixtures were run independently and proteins were visualized with Coomassie Brilliant Blue staining (lanes 4-6). The position of SUMO-1 and CBP is indicated by arrows. B. Peptide competition experiments. The following acetylated peptides were used: K25-K27:YIK(Ac)LK(Ac)VIG (lane 2); peptide K37-K39:HFK(Ac)VK(Ac)MTT (lane 3); peptide K45-K46-K48:HLK(Ac)K(Ac)LK(Ac)ES (lane 4). Lane 1 contains the acetylation reaction with a control peptide (QIRGRERFEM) and lane 5 contains the acetylation reaction without peptide(s). Polypeptides were incubated in a 10 fold excess relatively to full length SUMO-1. C. Identification of lysines residues acetylated in vitro and in vivo by mass spectrometry and alignment of acetylated residues in SUMO-1 with SUMO-2 and SUMO-3. Acetylated lysines of SUMO-1 are indicated by arrows and bolded in red, and conserved residues are shown. Based on the results of Mass Spectrometry we name the domain comprised between amino acid K37 to K48 SUMO-1 Acetylated Domain, or SAD. D. Acetylation of SUMO family members. Acetylation reactions were carried out as described in A. SUMO-1 (lanes 2, 5) SUMO-2 (lanes 3, 6), or SUMO-3 (lanes 4, 7) were subjected to an acetylation reaction in absence (lanes 2-4) or presence (lanes 5-7; indicated by +) of CBP. Lane 1 contains an acetylation reaction with CBP without SUMO proteins.

FIG. 3. SUMO-1 is acetylated when bound to its substrates. A. A polyclonal antibody raised against a SUMO-1 peptide SEIHF{Lys-Ac}VKMTTHLKKC acetylated at K37 was raised and purified by peptide affinity purification. The specificity of this antibody was tested first in an in vitro acetylation reaction similar to that described in FIG. 2A. SUMO-1 was incubated in the presence (lane 3) or absence (lane 2) of CBP and cold Acetyl-coenzyme A. Lane 1 contains the acetylation reaction with CBP alone without SUMO-1. Samples were subjected to SDS electrophoresis, blotted on a PDF membrane, and probed with Ac-K37-Ab in immunoblot (upper panel). After chemi-luminescence, the membrane was stained with Comassie Brilliant Blue to reveal SUMO-1 (lower panel). B. Acetylation of SUMO-1 in vivo. Control H1299 cells (lanes 1 and 4) or H1299 expressing naïve SUMO-1 (lanes 2 and 5) or a SUMO-1 harboring lysine to arginine replacements at position K37-K39 and K45-K46-K48 (SUMOK37-K48R, lanes 3 and 6) were induced with tetracycline. Cell extracts were absorbed on the anti-Flag M2 immuno-affinity column, eluted, and run in on 4-20% gradient gel. Following electrophoresis and transfer, membranes were immuno-blotted with the Ac-K37-Ab (left panel, lanes 1-3). Forty micrograms of total cell extracts were probed with the anti-Flag antibody in direct immuno-blot (right panel, lanes 4-6). The position of free SUMO-1 and of SUMO-1 conjugated to RanGap is indicated by arrows. C. Modulation of SUMO-1 acetylation by deacetylases and by DNA damaging agents. H1299 cells expressing SUMO-1 were left untreated (lanes 1 and 5) or they were treated with 500 nM TSA (lanes 2 and 6), with 1 μM etoposide (lanes 3 and 7), or with 100 mM H₂O₂ (lanes 4 and 8). Cell extracts were prepared in RIPA buffer, immuno-precipitated with the anti-Flag antibody and processed in immuno-blot with the Ac-K37-Ab as described in B (lanes 1-4). Total cell extracts are shown in the right panel. The position of SUMO-1, RanGaP and of SUMO-1 conjugates is indicated. Full arrows indicate non specific reactivity of the Ac-K37-Ab with heavy and light chains of the anti-Flag immuno-precipitation.

FIG. 4. SUMO-1 is acetylated when bound to p53 in murine tumors. A-C. Histopathology of a salivary gland pre-neoplastic dysplasia (PN) and a murine salivary adenocarcinoma (MSGT1-2). Open arrows indicate pre-neoplastic tissue and dark arrows indicate adenocarcinoma tissue. (D) Cell extracts prepared from PN and from murine salivary gland tumors (MSGT1-MSGT4) were immuno-precipitated with the anti-SUMO-1 monoclonal antibody and subsequently immuno-blotted with either anti-SUMO-1 (upper panel), anti-p53 goat polyclonal antibody (middle panel), or Ac-K37-Ab (lower panel). The position of sumoylated and acetylated p53 is indicated by arrows, and the position of these species relatively to the molecular weight reference marker is also shown. E. The total p53 levels as well as the expression levels of the p53 downstream targets p21 and 14-3-3-sigma along with the loading control actin, were determined by direct immuno-blot. Lane 5 is only apparently separate from the other samples, as it was run in the same immuno-blot, but a blank lane was cut in between.

FIG. 5. SAD is required for the apoptotic activity of sumoylated p53, but not for the cell cycle arrest function. A. Naïve H1299 cells (panels i and iv), or H1299 expressing SUMO-1 (panels ii and v) or SUMO-1K37-K48R (panels iii and vi) were infected with adenovirus control or with the adenovirus expressing p53 (panels i through iii and iv-through vi, respectively), as indicated at the top of each panel. 72 hours after infection, cells were harvested and their cell cycle profile was analyzed (see materials and methods). B-C. Analysis of the cell cycle profile of H1299 cells harboring p53-SUMO chimeric proteins indicated at the top of each panel. Cells in C were infected with control- or p53-expressing adenoviruses as described in A.

FIG. 6. A. H1299 cells expressing p53-SUMOΔGG (upper panels) or p53-SUMOΔGG^{K37-K48A} were plated onto cover-slips, induced with tetracycline for four days and processed in immuno-fluorescence. Cells were stained with the anti-p53 polyclonal antibody (left green panels) or with DAPI (middle blue panels), or merged (right panels). Z-stack images were acquired at 1-μm intervals, and best-focused areas are shown. Arrows in the DAPI staining point to apoptotic fragmented nuclei. B. Quantification of experiments shown in D. H1299 control cells, or cells expressing p53-SUMOΔGG or p53-SUMOΔGG^{K37-K48A} were processed for immuno-fluorescence as described in D. Apoptotic and fragmented nuclei were counted from a triplicate experiment and plotted. Bars represent standard deviations.

FIG. 7. A. SUMO-1 restricts p53-dependent transcription. A. Naïve H1299 (lanes 1 and 4), or cells harboring p53-SUMOΔGG (lanes 2 and 5) or p53-SUMOΔGG^{K37-K48A} (lanes 3 and 6), were infected with control- or p53-expressing adenoviruses (lanes 1-3 and lanes 4-6, respectively). Cell extracts derived from these cells were probed in direct immuno-blot with the anti-p53 polyclonal antibody, the p21 monoclonal antibody, the bax monoclonal antibody or the anti-actin antibody, as indicated at the side of each panel. The position of p53-SUMO proteins and of naïve p53 is indicated by arrows. B. Luciferase reporter assays were performed to assess how SUMO-1 influences the transcriptional activity of p53. The following vectors were employed for these experiments: the vector expressing naïve p53; or a p53 chimeric protein containing amino acid residues 14-to-55 of SUMO (p53-SAD); or SUMO^{K37-K48R}; or a vector expressing SUMO-1. Titration experiments were performed and plasmids were co-transected at the concentrations indicated at the left of the panel in the p53 null H1299 cell line with the luciferase reporter driven by the regulatory elements of the p21 promoter containing p53 DNA-binding sites. Luciferase activity was determined 24-36 hours after transfection. The type of p53 expressing vector is indicated at the bottom of each panel. Because transfection of SUMO-1 alone resulted in background transcriptional activity, values obtained in cells transfected with this vector were used for normalization, and considered as 1. At the bottom of the panels average fold activation after normalization is shown, for each p53 protein expressed at intermediate concentration of each plasmid (0.125 μg). C. Comparison of gene regulation between p53 and the p53-SUMO. Arrays were conducted as described in the materials and methods. A. Venn diagram representation of the total number of genes regulated by p53 and p53-SUMO, relatively to H1299 cells. Pie charts represent the number of genes either activated or repressed. D. Quantitative real-time PCR confirms gene targets activated by p53-SUMO as identified in the micro-array platform. cDNA generated from RNA extracted from parental H1299 cells or H1299 cells expressing p53 or p53-SUMO was used for analysis by SYBR green quantitative real-time PCR. Values were normalized against B-actin and fold activation was determined relatively to the parental H1299 sample. Each sample was performed in duplicate and the error bars represent the standard deviation.

FIG. 8. SUMO and SAD redistribute p53 on chromatin embedded p53-regulated promoters. A. Extracts derived from H1299 parental cells, or cells expressing p53, or p53-SUMO were subjected to chromatin immuno-precipitation with the anti-p53 polyclonal antibody. The DNA samples derived from each anti-p53 immuno-precipitation were amplified with pairs of primers directed to various p53-responsive target genes, as indicated on the right side of each panel. To normalize for DNA content, the DNA was amplified with the indicated primers prior to the anti-p53 immuno-precipitation (indicated as input at the top of each panel). B. Analysis of ChIP assays by densitometry. ChIP assays from cell lines indicated at the top of each panel, were conducted as described before. The primer pairs employed for the amplification reactions are indicated at the bottom of the panel. To quantify these experiments, densitometry of amplified bands was performed by using Adobe Photoshop. Readings were generated relatively to the signal obtained in H1299 cells and normalized against the input signal. Numbers represent average fold induction relatively to the H1299 control, normalized against the input from two PCR reactions. C. Chromatin immuno-precipitation assays were performed in naïve H1299 cells or cells expressing p53, p53-SUMO and SUMOK37-K48R by using primer pairs that amplify the p53-regulated promoters as indicated at the left side of each panel. All images shown were derived from the same gel, but in some cases lanes were cut in between samples.

FIG. 9. Molecular modeling of acetylation on the structure of SUMO-1. The x-ray crystal structure of SUMO-1 (PDB:1TGZ) was used for these simulations. Left panel (A), SUMO-1 is shown before acetylation, right panel (B), after acetylation. The SUMO1 structure with K37, K39, and K46 K48 is highlighted by ball and stick model, h-bond/salt-bridge shown by dotted line. The influence of acetylation of K37, K39, K46 and K48 of SUMO-1 was investigated. Molecular dynamics simulations demonstrated that inherent conformational changes are caused by lysine acetylation at all these residues. Acetylation of K48 alone tends to stabilize the structure, however in the case of surface residues such as K37, K39 and K46, the loss of positive charge upon acetylation causes the breakage of the inherent salt bridge interactions with the neighboring negatively charged residues.

### DETAILED DESCRIPTION OF THE INVENTION

The invention relates to antibodies or antibody fragments that specifically bind to an acetylation domain of a small ubiquitin-like modifier protein (SUMO protein) when the acetylation domain is at least partially acetylated.

Proteins belonging to the SUMO family (small ubiquitin-like modifiers) members belong to a group of post-translational modifiers that share similar three-dimensional fold with ubiquitin and are essential for transcription, DNA repair, cell survival and for genomic stability. There are four currently known members of the SUMO family, SUMO-1-4, each of which has been shown to play independent effects in regulating the activity of various intracellular proteins. SUMO-1 exists in the cells as a free, non conjugated form, as well as covalently conjugated to a variety of substrates.

The present invention relates to the discovery that SUMO proteins themselves are acetylated and that the state of acetylation of SUMO proteins directly regulates the activity of target factors that interact with SUMO. Thus, detection of acetylated SUMO proteins can be important for determining and understanding cellular responses where SUMO target proteins mediate the response. To that end, one aspect of the invention is directed to diagnostic methods, where the methods comprise determining the acetylation state of an acetylation domain within a SUMO protein in a sample of cells from a subject, wherein at least partial acetylation of the acetylated domain of the SUMO protein indicates that cells harboring the SUMO protein are susceptible to induction of cell death.

As used herein, the term SUMO protein is used, in general, to mean any SUMO protein, i.e., SUMO 1-4 from any species. For example, in one embodiment, the SUMO protein analyzed in the methods or towards which the antibody or antibody fragments are directed is SUMO-1 (SEQ ID NO:1), SUMO-2 (SEQ ID NO:2), SUMO-3 (SEQ ID NO:3), and/or SUMO-4 (SEQ ID NO:4). The SUMO proteins may be the mature form of the polypeptide or the propeptide form.

(SEQ ID NO: 1)
MSDQEAKPST EDLGDKKEGE YIKLKVIGQD SSEIHFKVKM TTHLKKLKES YCQRQGVPMN
SLRFLFEGQR IADNHTPKEL GMEEEDVIEV YQEQTGGHSV T
(SEQ ID NO: 2)
MADEKPKEGV KTENNDHINL KVAGQDGSVV QFKIKRHTPL SKLMKAYCER QGLSMRQIRF
RFDGQPINET DTPAQLEMED EDTIDVFQQQ TGGVY
(SEQ ID NO: 3)
MSEEKPKEGV KTENDHINLK VAGQDGSVVQ FKIKRHTPLS KLMKAYCERQ GLSMRQIRFR
FDGQPINETD TPAQLEMEDE DTIDVFQQQT GGVPESSLAG HSF
(SEQ ID NO: 4)
MANEKPTEEV KTENNNHINL KVAGQDGSVV QFKIKRQTPL SKLMKAYCEP RGLSMKQIRF
RFGGQPISGT DKPAQLEMED EDTIDVFQQP TGGVY

The mature form of human SUMO-1 is amino acid residues 1-97 of SEQ ID NO:1. The mature form of human SUMO-2 is amino acid residues 1-93 of SEQ ID NO:2. The mature form of human SUMO-3 is amino acid residues 1-92 of SEQ ID NO:3. The mature form of human SUMO-4 is amino acid residues 1-93 of SEQ ID NO:4.

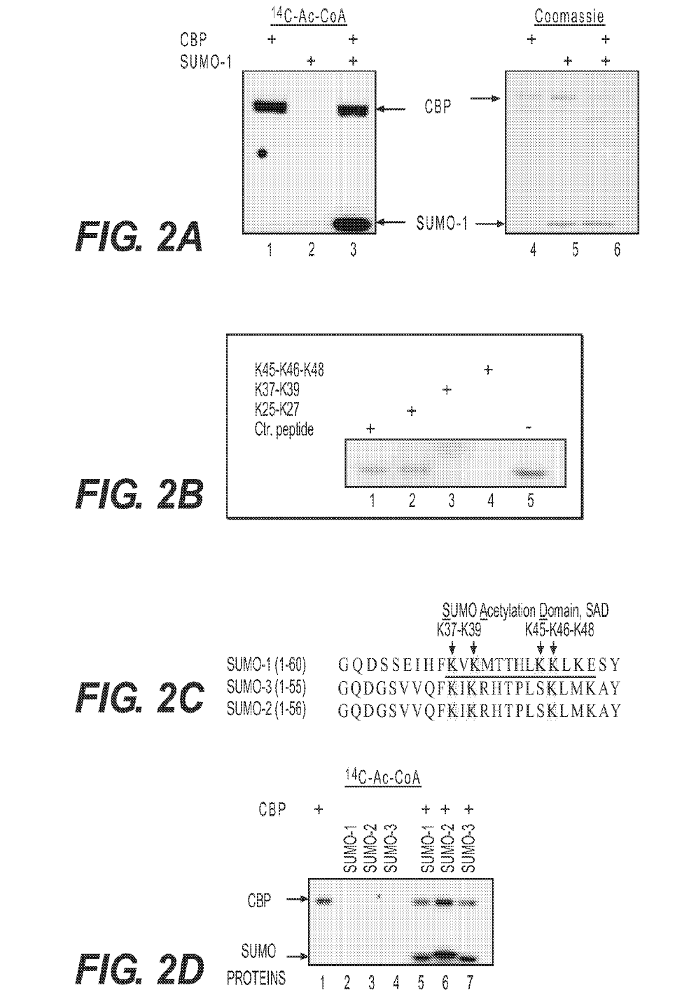

In every known SUMO protein, regardless of species, there exists an “acetylation domain.” As used herein, an acetylation domain of a SUMO protein is a stretch of amino acids containing 4 or 5 lysine residues that are highly conserved among each family member, i.e., SUMO 1-4, and also conserved across species, i.e., SUMO-1 across various mammalian species. FIG. 2C shows an alignment of the so-called SUMO acetylation domains across SUMO proteins 1-3. The acetylation domain of SUMO-1 corresponds to amino acid residues 37-48 of SEQ ID NO:1. The acetylation domain of SUMO-2 corresponds to amino acid residues 33-45 of SEQ ID NO:2. The acetylation domain of SUMO-3 corresponds to amino acid residues 32-44 of SEQ ID NO:3. The acetylation domain of SUMO-3 corresponds to amino acid residues 33-45 of SEQ ID NO:4. Accordingly, one aspect of the invention provides antibodies or antibody fragments that specifically bind acetylated polypeptides, where the amino acid sequence of the acetylated polypeptides comprises the acetylation domains of SEQ ID NO:1, 2, 3 or 4. Another aspect of the invention relates to detecting the acetylation state of acetylation domains comprising the acetylation domains of SEQ ID NO:1, 2, 3 or 4.

As used herein, “acetylated SUMO protein” (or, in general “acetylated polypeptide” or “acetylated protein”) indicates that at least one lysine residue on the SUMO protein is acetylated. Likewise, an “acetylated domain,” e.g., acetylation domain, indicates that at least one lysine residue within the domain is acetylated. The acetylated SUMO protein or domains thereof may comprise more than one acetylated lysine and may include a SUMO protein or domain where all lysine residues in the protein or domain are acetylated (“fully acetylated”). As used herein, the phrase “partial acetylation” or “partially acetylated” is used to mean a SUMO protein or an acetylation domain thereof with at least one lysine residue acetylated, but less than every lysine residue in the polypeptide or domain being acetylated. The “acetylation state” of a protein or an acetylation domain thereof is used to mean a protein or domain thereof that is at least partially acetylated, completely acetylated (all lysine residues are acetylated) or unacetylated (no lysine residues are acetylated).

One embodiment of the invention provides antibodies or antibody fragments that specifically bind acetylated polypeptides, where the amino acid sequence of the acetylated polypeptides comprises the amino acid sequence of SEQ ID NO:1, 2, 3 or 4. Another aspect of the invention relates to detecting the acetylation state of acetylation domains comprising the amino acid sequence of SEQ ID NO:1, 2, 3 or 4.

Another embodiment of the invention provides antibodies or antibody fragments that specifically bind acetylated polypeptides, where the amino acid sequence of the acetylated polypeptides comprises amino acid residues 1-97 of SEQ ID NO:1. Another embodiment of the invention provides antibodies or antibody fragments that specifically bind acetylated polypeptides, where the amino acid sequence of the acetylated polypeptides comprises amino acid residues 1-93 of SEQ ID NO:2. Another embodiment of the invention provides antibodies or antibody fragments that specifically bind acetylated polypeptides, where the amino acid sequence of the acetylated polypeptides comprises amino acid residues 1-92 of SEQ ID NO:3. Another embodiment of the invention provides antibodies or antibody fragments that specifically bind acetylated polypeptides, where the amino acid sequence of the acetylated polypeptides comprises amino acid residues 1-93 of SEQ ID NO:4.

Another embodiment of the invention provides antibodies or antibody fragments that specifically bind acetylated polypeptides, where the amino acid sequence of the acetylated polypeptides consists of amino acid residues 1-97 of SEQ ID NO:1. Another embodiment of the invention provides antibodies or antibody fragments that specifically bind acetylated polypeptides, where the amino acid sequence of the acetylated polypeptides consists of amino acid residues 1-93 of SEQ ID NO:2. Another embodiment of the invention provides antibodies or antibody fragments that specifically bind acetylated polypeptides, where the amino acid sequence of the acetylated polypeptides consists of amino acid residues 1-92 of SEQ ID NO:3. Another embodiment of the invention provides antibodies or antibody fragments that specifically bind acetylated polypeptides, where the amino acid sequence of the acetylated polypeptides consists of amino acid residues 1-93 of SEQ ID NO:4.

One embodiment of the invention provides antibodies or antibody fragments that specifically bind acetylated polypeptides, where the amino acid sequence of the acetylated polypeptides consists of the amino acid sequence of SEQ ID NO:1, 2, 3 or 4. Another aspect of the invention relates to detecting the acetylation state of acetylation domains comprising the amino acid sequence of SEQ ID NO:1, 2, 3 or 4.

The invention further embraces other species, preferably mammalian, homologs with amino acid sequences that correspond to the SUMO proteins and acetylation domains thereof. Species homologs, sometimes referred to as “orthologs,” in general, share at least 35%, 40%, 45%, 50%, 60%, 65%, 70%, 75%, 80%, 85%, 90%, 95%, 96%, 97%, 98%, or 99% sequence identity with the human version of each of the SUMO proteins. Such corresponding sequences account for SUMO proteins across a variety of species, such as canine, feline, mouse, rat, rabbit, monkey, etc.

As used herein, the terms “correspond(s) to” and “corresponding to,” as they relate to sequence alignment, are intended to mean enumerated positions within the reference protein, e.g., human SUMO-1, and those positions in orthologs or homologs that align with the positions on the reference protein. Thus, when the amino acid sequence of a subject SUMO-1 is aligned with the amino acid sequence of a reference SUMO-1, e.g., SEQ ID NO:1, the amino acids in the subject sequence that “correspond to” certain enumerated positions of the reference sequence are those that align with these positions of the reference sequence, e.g., SEQ ID NO:1, but are not necessarily in these exact numerical positions of the reference sequence. Methods for aligning sequences for determining corresponding amino acids between sequences are described herein.

Accordingly, the invention provides antibodies or antibody fragments that specifically bind acetylated polypeptides, where the amino acid sequence of the acetylated polypeptides corresponds to the acetylation domains of SEQ ID NO:1, 2, 3 or 4. The invention also relates to detecting the acetylation state of acetylation domains corresponding to acetylation domains of SEQ ID NO:1, 2, 3 or 4. In one embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to amino acid residues 37-48 of SEQ ID NO:1. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 30-50 of SEQ ID NO:1. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to amino acid residues 33-45 of SEQ ID NO:2. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to amino acid residues 32-44 of SEQ ID NO:3. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to amino acid residues 33-45 of SEQ ID NO:4.

In another embodiment, the antibodies or antibody fragments of the present invention specifically bind to other acetylation sites of an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 1-20 of SEQ ID NO:1. In one embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 7-27 of SEQ ID NO:1. In one embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 27-40 of SEQ ID NO:1. In one embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 10-21 of SEQ ID NO:1. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, 99% or 100% identical to amino acid residues 40-58 of SEQ ID NO:1.

In an additional embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to amino acid sequence of SEQ ID NO:1. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to the amino acid sequence of SEQ ID NO:2. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to the amino acid sequence of SEQ ID NO:3. In another embodiment, the antibodies or antibody fragments of the present invention specifically bind to an acetylated polypeptide, wherein the polypeptide comprises an amino acid sequence at least 60%, 65%, 70%, 75%, 80%, 85%, 90%, 95%, 96%, 97%, 98%, or 99% identical to the amino acid sequence of SEQ ID NO:4.

In an additional embodiment, the antibodies or antibody fragments of the present invention specifically bind to an acetylated polypeptide, wherein the polypeptide consists of an amino acid sequence at least 60%, 65%, 70%, 75%, 80%, 85%, 90%, 95%, 96%, 97%, 98%, or 99% identical to SEQ ID NO:1. In another embodiment, the antibodies or antibody fragments of the present invention specifically bind to an acetylated polypeptide, wherein the polypeptide consists of an amino acid sequence at least 60%, 65%, 70%, 75%, 80%, 85%, 90%, 95%, 96%, 97%, 98%, or 99% identical to SEQ ID NO:2. In another embodiment, the antibodies or antibody fragments of the present invention specifically bind to an acetylated polypeptide, wherein the polypeptide consists of an amino acid sequence at least 60%, 65%, 70%, 75%, 80%, 85%, 90%, 95%, 96%, 97%, 98%, or 99% identical to SEQ ID NO:3. In another embodiment, the antibodies or antibody fragments of the present invention specifically bind to an acetylated polypeptide, wherein the polypeptide consists of an amino acid sequence at least 60%, 65%, 70%, 75%, 80%, 85%, 90%, 95%, 96%, 97%, 98%, or 99% identical to SEQ ID NO:4.

A polypeptide having an amino acid sequence at least, for example, about 95% “identical” to a reference an amino acid sequence, e.g., SEQ ID NO:1, is understood to mean that the amino acid sequence of the polypeptide is identical to the reference sequence except that the amino acid sequence may include up to about five modifications per each 100 amino acids of the reference amino acid sequence. In other words, to obtain a polypeptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While there are several methods to measure identity between two polynucleotide or polypeptide sequences, the term “identity” is well known to skilled artisans (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCG program package (Devereux, J., et al., Nucleic Acids Research 12(i):387 (1984)), BLASTP, ExPASy, BLASTN, FASTA (Atschul, S. F., et al., J Molec Biol 215:403 (1990)) and FASTDB. Examples of methods to determine identity and similarity are discussed in Michaels, G. and Garian, R., Current Protocols in Protein Science, Vol 1, John Wiley & Sons, Inc. (2000), which is incorporated by reference.

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag et al. (Comp. App. Biosci. 6:237-245 (1990), incorporated by reference). In a FASTDB sequence alignment, the query and reference sequences are amino sequences. The result of sequence alignment is in percent identity. In one embodiment, parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score can be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment−10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

The acetylated SUMO proteins or acetylated acetylation domains thereof, or fragments thereof or other derivatives, or analogs thereof, or cells expressing them can be used as an immunogen to produce antibodies or antibody fragments thereof. Any of the antibodies can be, for example, polyclonal, monoclonal, bi-specific, multispecific, human or chimeric antibodies. The antibody molecules of the invention can be of any type, e.g., IgG, IgE, IgM, IgD, IgA and IgY, class, e.g., IgG1, IgG2, IgG3, IgG4, IgA1 and IgA2 or subclass of immunoglobulin molecule. In one embodiment, an antibody of the invention comprises, or alternatively consists of, a polypeptide having an amino acid sequence of a VH domain, at least one VH CDR, a VL domain, or at least one VL CDR.

The antibodies or antibody fragments of the present invention may be monovalent, bivalent, trivalent or multivalent. For example, monovalent scFvs can be multimerized either chemically or by association with another protein or substance. An scFv that is fused to a hexahistidine tag or a Flag tag can be multimerized using Ni-NTA agarose (Qiagen) or using anti-Flag antibodies (Stratagene, Inc.).

The antibodies of the present invention may be monospecific, bispecific, trispecific or of greater multispecificity. Multispecific antibodies may be specific for different epitopes of an acetylated SUMO protein, or a domain thereof, or may be specific for both an acetylated SUMO protein, or a domain thereof, and a heterologous epitope, such as a heterologous polypeptide or solid support material. See, e.g., PCT publications WO 93/17715; WO 92/08802; WO 91/00360; WO 92/05793; Tutt, et al, J. Immunol. 147:60 69 (1991); U.S. Pat. Nos. 4,474,893; 4,714,681; 4,925,648; 5,573,920; 5,601,819; Kostelny et al, J. Immunol. 148:1547-1553 (1992), which are incorporated by reference.

As used herein, an antibody fragment is a fragment of an antibody capable of specifically binding the same epitope that the intact antibody would bind. Examples of antibody fragments include but are not limited to Fab and F(ab′)₂ fragments, Fd fragments, disulfide-linked Fvs (sd Fvs), antiidiotypic (anti-Id) antibodies, including but not limited to anti-Id antibodies to antibodies of the invention, and epitope-binding fragments of any of the above. Fab and F(ab′)₂ fragments lack the Fc fragment of intact antibody and generally clear more rapidly from the circulation, and may have less non-specific tissue binding than that of an intact antibody (Wahl et al., J. Nucl. Med. 24:316-325 (1983)). Other types of antibody fragments include but are not limited to single chain Fv fragments (scFv) that are well-known in the art. Techniques described for the production of single chain antibodies (U.S. Pat. No. 4,946,778) can be adapted to produce single chain antibodies to immunogenic polypeptide products of this invention.

The antibodies or fragments of the present invention may be prepared by any of a variety of methods. For example, cells expressing acetylated SUMO protein or an antigenic fragment thereof can be administered to an animal to induce the production of sera containing polyclonal antibodies. In one method, a preparation of SUMO-1, SUMO-2, SUMO-3 or SUMO-4 protein is prepared and purified to render it substantially free of natural contaminants. Such a preparation is then introduced into an animal in order to produce polyclonal antisera of greater specific activity.

Accordingly, one aspect of the invention provides a method for making acetylation site-specific antibodies.

Polyclonal antibodies of the invention may be produced according to standard techniques by immunizing a suitable animal, e.g., rabbit, goat, etc., with an antigen comprising a novel lysine acetylation site of the invention. Antibodies can be produced that are specific for either the acetylated or unacetylated state, depending upon the desired specificity of the antibody. Collecting immune serum from the animal and separating the polyclonal antibodies from the immune serum can be carried out in accordance with known procedures, and screening and isolating a polyclonal antibody specific for the novel lysine acetylation site of interest can be carried out with well-known procedures and as described below. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990, which is incorporated by reference.

The immunogen may be the full length protein or a peptide comprising a lysine acetylation site of interest, such as, but not limited to an acetylation domain. In some embodiments the immunogen is a peptide of from 7 to 20 amino acids in length, in particular from about 8 to 17 amino acids in length. In some embodiments, the peptide antigen desirably will comprise about 3 to 8 amino acids on each side of the phosphorylatable lysine. In yet other embodiments, the peptide antigen desirably will comprise four or more amino acids flanking each side of the phosphorylatable amino acid and encompassing it. Peptide antigens suitable for producing antibodies of the invention may be designed, constructed and employed in accordance with well-known techniques. See, e.g., Antibodies: A Laboratory Manual, Chapter 5, p. 75-76, Harlow & Lane Eds., Cold Spring Harbor Laboratory (1988); Czemik, Methods In Enzymology, 201: 264-283 (1991); Merrifield, J. Am. Chem. Soc. 85: 21-49 (1962), which are incorporated by reference.

In some embodiments the immunogen is administered with an adjuvant. Suitable adjuvants will be well known to those of skill in the art. Exemplary adjuvants include complete or incomplete Freund's adjuvant, RIBI (muramyl dipeptides) or ISCOM (immunostimulating complexes).

When the above-described methods are used for producing polyclonal antibodies, following immunization, the polyclonal antibodies which secreted into the bloodstream can be recovered using known techniques. Purified forms of these antibodies can, of course, be readily prepared by standard purification techniques, such as for example, affinity chromatography with Protein A, anti-immunoglobulin, or the antigen itself. In any case, to monitor the success of immunization, the antibody levels with respect to the antigen in serum can be monitored using standard techniques such as ELISA, RIA and the like.

In one aspect of the present invention, the antibodies or fragments thereof of the present invention are monoclonal antibodies. Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., (1981) pp. 563-681). In general, such procedures involve immunizing an animal (for example a mouse) with an acetylated SUMO-1, SUMO-2, SUMO-3 or SUMO-4 protein antigen or with a SUMO-1, SUMO-2, SUMO-3 or SUMO-4 protein-expressing cell. Suitable cells can be recognized by their capacity to bind anti-SUMO-1 (acetylated), anti-SUMO-2 (acetylated), anti-SUMO-3 (acetylated) or anti-SUMO-4 (acetylated) protein antibody. Such cells may be cultured in any suitable tissue culture medium; however, it is desirable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is may be desirable to employ the parent myeloma cell line (SP₂O), available from the American Type Culture Collection, Rockville, Md. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding acetylated SUMO-1, SUMO-2, SUMO-3 or SUMO-4 protein antigen. The secreted antibody may be recovered from tissue culture supernatant by conventional methods such as precipitation, ion exchange or affinity chromatography, or the like. Other methods of generating monoclonal antibodies include but are not limited to the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Alternatively, additional antibodies capable of binding to acetylated SUMO-1, SUMO-2, SUMO-3 or SUMO-4 protein antigen may be produced in a two-step procedure through the use of anti-idiotypic antibodies. Such a method makes use of the fact that antibodies are themselves antigens, thus it is possible to obtain an antibody which binds to a second antibody. In accordance with this method, acetylated SUMO-1, SUMO-2, SUMO-3 or SUMO-4 protein specific antibodies are used to immunize an animal, for example a mouse. The splenocytes of such an animal are then used to produce hybridoma cells, and the hybridoma cells are screened to identify clones which produce an antibody whose ability to bind to an acetylated SUMO-1, SUMO-2, SUMO-3 or SUMO-4 protein-specific antibody can be blocked by an acetylated SUMO-1, SUMO-2, SUMO-3 or SUMO-4 protein antigen, respectively. Such antibodies comprise anti-idiotypic antibodies to an acetylated SUMO-1, SUMO-2, SUMO-3 or SUMO-4 protein-specific antibody and can be used to immunize an animal to induce formation of acetylated SUMO-1, SUMO-2, SUMO-3 or SUMO-4 protein-specific antibodies.

The invention also encompasses antibody-producing cells and cell lines, such as hybridomas, as described above.

Polyclonal or monoclonal antibodies may also be obtained through in vitro immunization. For example, phage display techniques can be used to provide libraries containing a repertoire of antibodies with varying affinities for a particular antigen. Techniques for the identification of high affinity human antibodies from such libraries are described by Griffiths et al., EMBO J., 13:3245-3260 (1994); which is incorporated by reference.

The antibodies may be produced recombinantly using methods well known in the art for example, according to the methods disclosed in U.S. Pat. No. 4,349,893 or U.S. Pat. No. 4,816,567, which are incorporated by reference. The antibodies may also be chemically constructed by specific antibodies made according to the method disclosed in U.S. Pat. No. 4,676,980, which is incorporated by reference.

Once a desired acetylation site-specific antibody is identified, polynucleotides encoding the antibody, such as heavy, light chains or both (or single chains in the case of a single chain antibody) or portions thereof such as those encoding the variable region, may be cloned and isolated from antibody-producing cells using means that are well known in the art. For example, the antigen combining site of the monoclonal antibody can be cloned by PCR and single-chain antibodies produced as phage-displayed recombinant antibodies or soluble antibodies in E. coli. See, e.g., Antibody Engineering Protocols, Humana Press, Sudhir Paul, Ed. (1995), which is incorporated by reference.

Accordingly, in a further aspect, the invention provides such nucleic acids encoding the heavy chain, the light chain, a variable region, a framework region or a CDR of an antibody of the invention. In some embodiments, the nucleic acids are operably linked to expression control sequences. The invention, thus, also provides vectors and expression control sequences useful for the recombinant expression of an antibody or antigen-binding portion thereof of the invention. Those of skill in the art will be able to choose vectors and expression systems that are suitable for the host cell in which the antibody or antigen-binding portion is to be expressed.

In one embodiment, a nucleic acid molecule of the invention encodes an antibody comprising, or alternatively consisting of, a VH domain having an amino acid sequence of any one of the VH domains of the antibodies described herein. In another embodiment, a nucleic acid molecule of the present invention encodes an antibody comprising, or alternatively consisting of, a VH CDR1 having an amino acid sequence of any of the antibodies described herein. In another embodiment, a nucleic acid molecule of the present invention encodes an antibody comprising, or alternatively consisting of, a VH CDR2 having an amino acid sequence of any one of the VH CDR2 of any of the antibodies described herein. In yet another embodiment, a nucleic acid molecule of the present invention encodes an antibody comprising, or alternatively consisting of, a VH CDR3 having an amino acid sequence of any of the antibodies described herein. Nucleic acid molecules encoding antibodies that immunospecifically bind acetylated SUMO and comprise, or alternatively consist of, fragments or variants of the VH domains and/or VH CDRs are also encompassed by the invention.

In another embodiment, a nucleic acid molecule of the invention encodes an antibody, including molecules comprising, or alternatively consisting of, antibody fragments or variants thereof, comprising, or alternatively consisting of, a VL domain having an amino acid sequence of any one of the VL, domains of any of the antibodies described herein. In another embodiment, a nucleic acid molecule of the present invention encodes an antibody comprising, or alternatively consisting of, a VL CDR1 having amino acid sequence of any one of the any of the antibodies described herein. In another embodiment, a nucleic acid molecule of the present invention encodes an antibody comprising, or alternatively consisting of, a VL CDR2 having an amino acid sequence of any one of the VL CDR2 of any of the antibodies described herein. In yet another embodiment, a nucleic acid molecule of the present invention encodes an antibody comprising, or alternatively consisting of, a VL CDR3 having an amino acid sequence of any, one of the VL CDR3 of any of the antibodies described herein. Nucleic acid encoding antibodies that immunospecifically bind acetylated SUMO and comprise, or alternatively consist of, fragments or variants of the VL domains and/or VLCDR(s) are also encompassed by the invention.

In another embodiment, a nucleic acid molecule of the invention encodes an antibody comprising, or alternatively consisting of, a VH domain having an amino acid sequence of any one of the VH domains of any of the antibodies described herein, and a VL domain having an amino acid sequence of any one of the VL domains of any of the antibodies described herein. In another embodiment, a nucleic acid molecule of the invention encodes an antibody comprising, or alternatively consisting of, a VH CDR1, a VL CDR1, a VH CDR2, a VL CDR2, a VH CDR3, a VL CDR3, or any combination thereof having an amino acid sequence of any of the antibodies described herein. Nucleic acid encoding antibodies that immunospecifically bind acetylated SUMO and comprise, or alternatively consist of, fragments or variants of the VL and/or domains and/or VHCDR(s) and/or VLCDR(s) are also encompassed by the invention.

The present invention also provides antibodies that comprise, or alternatively consist of, variants, including derivatives, of the VH domains, VH CDRs, VL domains, and VL CDRs described herein, which antibodies immunospecifically bind to acetylated SUMO. Standard techniques known to those of skill in the art can be used to introduce mutations in the nucleotide sequence encoding a molecule of the invention, including, for example, site-directed mutagenesis and PCR-mediated mutagenesis which result in amino acid substitutions. In select embodiment, the variants, including derivatives, encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. In specific embodiments, the variants encode substitutions of VHCDR3. In a preferred embodiment, the variants have conservative amino acid substitutions at one or more predicted non-essential amino acid residues. A “conservative amino acid substitution” is one in which the amino acid residue is replaced with an amino acid residue having a side chain with a similar charge. Conservative substitutions are shown in the Tables below. Alternatively, mutations can be introduced randomly along all or part of the coding sequence, such as by saturation mutagenesis, and the resultant mutants can be screened for biological activity to identify mutants that retain activity, e.g., the ability to bind acetylated SUMO. Following mutagenesis, the encoded protein may routinely be expressed and the functional and/or biological activity of the encoded protein, e.g., ability to immunospecifically bind acetylated SUMO, can be determined using techniques described herein or by routinely modifying techniques known in the art.

TABLE I
Conservative Substitutions

| SIDE CHAIN CHARACTERISTIC | AMINO ACID | | | |
|---|---|---|---|---|
| Aliphatic | | | | |
| Non-polar | G | A | P | |
| | I | L | V | |
| Polar - uncharged | C | S | T | M |
| | N | Q | | |
| Polar - charged | D | E | | |
| | K | R | | |
| Aromatic | H | F | W | Y |
| Other | N | Q | D | E |

Alternatively, conservative amino acids can be grouped as described in Lehninger, [Biochemsitry, Second Edition; Worth Publishers, Inc. NY, N.Y. (1975), pp. 71 77] as set out below.

TABLE II
Conservative Substitutions

| SIDE CHAIN CHARACTERISTIC | AMINO ACID | | | | |
|---|---|---|---|---|---|
| Non-polar (hydrophobic) | | | | | |
| A. Aliphatic: | A | L | I | V | P |
| B. Aromatic: | F | W | | | |
| C. Sulfur-containing: | M | | | | |
| D. Borderline: | G | | | | |
| Uncharged-polar | | | | | |
| A. Hydroxyl: | S | T | Y | | |
| B. Amides: | N | Q | | | |
| C. Sylfhydryl: | C | | | | |
| D. Borderline: | G | | | | |
| Positively Charged (Basic): | K | R | H | | |
| Negatively Charged (Acidic) | D | E | | | |

And still other alternative, exemplary conservative substitutions are set out below.

TABLE III
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

The antibodies of the invention include derivatives or variants that are modified, e.g., by the covalent attachment of any type of molecule to the antibody such that covalent attachment does not affect the ability of the antibody to immunospecifically bind to acetylated SUMO. For example, but not by way of limitation, derivatives of the invention include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not, limited to, specific chemical cleavage, acetylation, formulation, metabolic synthesis of tunicamycin, etc. Additionally, the derivative may contain one or more non-classical amino acids.

In a specific embodiment, an antibody or antibody fragment of the invention, including a molecule comprising, or alternatively consisting of, an antibody fragment or variant thereof, that immunospecifically binds acetylated SUMO, comprises, or alternatively consists of, an amino acid sequence encoded by a nucleotide sequence that hybridizes to a nucleotide sequence that is complementary to that encoding one of the VH or VL domains of any of the antibodies described herein under stringent conditions, e.g., hybridization to filter-bound DNA in 6× sodium chloride/sodium citrate (SSQ at about 45° C. followed by one or more washes in 0.2×SSC/0.1% SDS at about 50°-65° C., under highly stringent conditions, e.g., hybridization to filter-bound nucleic acid in 6×SSC at about 45° C., followed by one or more washes in 0.1×SSC/0.2% SDS at about 68° C., or under other stringent hybridization conditions which are known to those of skill in the art.

In another embodiment, an antibody or antibody fragment of the invention that immunospecifically binds to acetylated SUMO, comprises, or alternatively consists of, an amino acid sequence encoded by a nucleotide sequence that hybridizes to a nucleotide sequence that is complementary to that encoding one of the VH CDRs or VL CDRs of any of the antibodies described herein under stringent conditions, e.g., hybridization under conditions as described above, or under other stringent hybridization conditions which are known to those of skill in the art.

In another embodiment, an antibody of antibody fragment that immunospecifically binds to acetylated SUMO comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH domains of any of the antibodies described herein. In another embodiment, the invention provides an antibody or antibody fragment of the invention that immunospecifically binds to acetylated SUMO comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH CDRs of any of the antibodies described herein. Nucleic acid molecules encoding these antibodies are also encompassed by the invention.

In another embodiment, an antibody or antibody fragment of the invention that immunospecifically binds to acetylated SUMO comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL domains of any of the antibodies described herein. In another embodiment, the invention provides an antibody of the invention that immunospecifically binds to acetylated SUMO comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL CDRs of any of the antibodies described herein.

In yet another embodiment, the invention provides antibodies or antibody fragments that immunospecifically binds to acetylated SUMO comprising, or alternatively consisting of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to at least one, two or three of the VH CDRs of any of the antibodies described herein and that is at least 35%, at least 40%, at least 45%, at least 50%, at least 55%, at least 60%, at least 65%, at least 70%, at least 75%, at least 80%, at least 85%, at least 90%, at least 95%, or at least 99% identical to at least one, two or three of the VL CDRs of any of the antibodies described herein. Of course, the invention also provides antibodies or antibody fragments that immunospecifically binds to acetylated SUMO comprising, or alternatively consisting of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to at least two or three of the VH CDRs of any of the antibodies described herein and that is at least 35%, at least 40%, at least 45%, at least 50%, at least 55%, at least 60%, at least 65%, at least 70%, at least 75%, at least 80%, at least 85%, at least 90%, at least 95%, or at least 99% identical to at least two or three of the VL CDRs of any of the antibodies described herein. The invention also provides antibodies or antibody fragments that immunospecifically binds to acetylated SUMO comprising, or alternatively consisting of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to three of the VH CDRs of any of the antibodies described herein and that is at least 35%, at least 40%, at least 45%, at least 50%, at least 55%, at least 60%, at least 65%, at least 70%, at least 75%, at least 80%, at least 85%, at least 90%, at least 95%, or at least 99% identical to three of the VL CDRs of any of the antibodies described herein. Nucleic acid molecules encoding these antibodies are also encompassed by the invention.

Antibodies or fragments of the present invention may also be described or specified in terms of their binding affinity for to acetylated SUMO or domains or variants of acetylated SUMO.

In specific embodiments, antibodies or fragments of the invention bind acetylated SUMO or domains or variants thereof, with a dissociation constant or K_{d} of less than or equal to 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M or 10⁻¹⁵ M.

Monoclonal Fab fragments may also be produced in Escherichia coli by recombinant techniques known to those skilled in the art. See, e.g., W. Huse, Science 246: 1275-81 (1989); Mullinax et al., Proc. Nat'l. Acad. Sci. 87: 8095 (1990).

If monoclonal antibodies of a single desired isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)). Alternatively, the isotype of a monoclonal antibody with desirable propertied can be changed using antibody engineering techniques that are well-known in the art.

Acetylation site-specific antibodies of the invention, whether polyclonal or monoclonal, may be screened for epitope and phospho-specificity according to standard techniques. See, e.g., Czernik et al., Methods in Enzymology, 201: 264-283 (1991). For example, the antibodies may be screened against the acetylated and/or unacetylated peptide library by ELISA to ensure specificity for both the desired antigen, i.e., the epitope including an acetylation site of the invention, and for reactivity only with the acetylated (or unacetylated) form of the antigen. Peptide competition assays may be carried out to confirm lack of reactivity with other phospho-epitopes on the parent protein. The antibodies may also be tested by Western blotting against cell preparations containing the parent signaling protein, e.g., cell lines over-expressing the parent protein, to confirm reactivity with the desired acetylated epitope/target.

Specificity against the desired acetylated epitope may also be examined by constructing mutants lacking phosphorylatable residues at positions outside the desired epitope that are known to be acetylated, or by mutating the desired phospho-epitope and confirming lack of reactivity. Acetylation site-specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. Cross-reactivity with non-target proteins is readily characterized by Western blotting alongside markers of known molecular weight. Amino acid sequences of cross-reacting proteins may be examined to identify acetylation sites with flanking sequences that are highly homologous to that of an acetylation site of the invention.

In certain cases, polyclonal antisera may exhibit some undesirable general cross-reactivity to acetyl-lysine itself, which may be removed by further purification of antisera, e.g., over an acetyl-lysine column. Antibodies of the invention specifically bind their target protein only when acetylated (or only when not acetylated, as the case may be) at the site disclosed herein and do not substantially or specifically bind to the other forms, as compared to the form for which the antibody is specific.

Antibodies may be further characterized via immunohistochemical (IHC) staining using normal and diseased tissues to examine acetylation and activation state and level of an acetylation site in diseased tissue. IHC may be carried out according to well-known techniques. See, e.g., Antibodies: A Laboratory Manual, Chapter 10, Harlow & Lane Eds., Cold Spring Harbor Laboratory (1988). Briefly, paraffin-embedded tissue, e.g., tumor tissue, is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry, Communications in Clinical Cytometry 46: 72-78 (2001). Briefly and by way of example, the following protocol for cytometric analysis may be employed: samples may be centrifuged on Ficoll gradients to remove lysed erythrocytes and cell debris. Adhering cells may be scraped off plates and washed with PBS. Cells may then be fixed with 2% paraformaldehyde for 10 minutes at 37° C. followed by permeabilization in 90% methanol for 30 minutes on ice. Cells may then be stained with the primary acetylation site-specific antibody of the invention, which can detect an acetylated SUMO protein, washed and labeled with a fluorescent-labeled secondary antibody. Additional fluorochrome-conjugated marker antibodies, e.g., CD45, CD34, may also be added at this time to aid in the subsequent identification of specific hematopoietic cell types. The cells would then be analyzed on a flow cytometer, e.g., a Beckman Coulter FC500, according to the specific protocols of the instrument used.

Antibodies of the invention may also be conjugated to fluorescent dyes, e.g., Alexa488, PE, etc., for use in multi-parametric analyses along with other signal transduction, e.g., phospho-CrkL, phospho-Erk 1/2, and/or cell marker, e.g., CD34 antibodies.

Acetylation site-specific antibodies of the invention may specifically bind to an acetylated SUMO protein or an acetylation domain of a SUMO protein only when acetylated at the specified lysine residue. The antibodies described herein are not necessarily limited only to binding to the specific acetylation site of the SUMO protein.

Methods for making bispecific antibodies are within the purview of those skilled in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Antibody variable domains with the desired binding specificities (antibody-antigen combining sites) can be fused to immunoglobulin constant domain sequences. In certain embodiments, the fusion is with an immunoglobulin heavy-chain constant domain, including at least part of the hinge, CH2, and CH3 regions. DNAs encoding the immunoglobulin heavy-chain fusions and, if desired, the immunoglobulin light chain, are inserted into separate expression vectors, and are co-transfected into a suitable host organism. For further details of illustrative currently known methods for generating bispecific antibodies see, for example, Suresh et al., Methods in Enzymology, 121:210 (1986); WO 96127011; Brennan et al., Science 229:81 (1985); Shala by et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); and Tutt et al., J. Immunol. 147:60 (1991). Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. A strategy for making bispecific antibody fragments by the use of single-chain Fv (scFv) dimers has also been reported. See Gruber at al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H1}-V_{H}-C_{H1}) which form a pair of antigen binding regions. Linear antibodies can be bispecific or monospecific. To produce the chimeric antibodies, the portions derived from two different species, e.g., human constant region and murine variable or binding region can be joined together chemically by conventional techniques or can be prepared as single contiguous proteins using genetic engineering techniques. The DNA molecules encoding the proteins of both the light chain and heavy chain portions of the chimeric antibody can be expressed as contiguous proteins. The method of making chimeric antibodies is disclosed in U.S. Pat. No. 5,677,427; U.S. Pat. No. 6,120,767; and U.S. Pat. No. 6,329,508 which are incorporated by reference.

Fully human antibodies may be produced by a variety of techniques. One example is trioma methodology. The basic approach and an exemplary cell fusion partner, SPAZ-4, for use in this approach have been described by Oestberg et al., Hybridoma 2:361-367 (1983); Oestberg, U.S. Pat. No. 4,634,664; and U.S. Pat. No. 4,634,666, which are incorporated by reference.

Human antibodies can also be produced from non-human transgenic animals having transgenes encoding at least a segment of the human immunoglobulin locus. The production and properties of animals having these properties are described in detail by, see, e.g., WO93/12227; U.S. Pat. No. 5,545,806, WO91/10741 and U.S. Pat. No. 6,150,584, which are herein incorporated by reference.

Various recombinant antibody library technologies may also be utilized to produce fully human antibodies. For example, one approach is to screen a DNA library from human B cells according to the general protocol outlined by Huse et al., Science 246:1275-1281 (1989). The protocol described by Huse is rendered more efficient in combination with phage-display technology. See, WO 91/17271, WO 92/01047 and U.S. Pat. No. 5,969,108, which are incorporated by reference.

Eukaryotic ribosome can also be used as means to display a library of antibodies and isolate the binding human antibodies by screening against the target antigen, as described in Coia G, et al., J. Immunol. Methods 1: 254 (1-2):191-7 (2001); Hanes J. et al., Nat. Biotechnol. 18(12):1287-92 (2000); Proc. Natl. Acad. Sci. U.S.A. 95(24):14130-5 (1998); Proc. Natl. Acad. Sci. U.S.A. 94(10):4937-42 (1997), which are incorporated by reference.

The yeast system is also suitable for screening mammalian cell-surface or secreted proteins, such as antibodies. Antibody libraries may be displayed on the surface of yeast cells for the purpose of obtaining the human antibodies against a target antigen. This approach is described by Yeung, et al., Biotechnol. Prog. 18(2):212-20 (2002); Boeder, E. T., et al., Nat. Biotechnol. 15(6):553-7 (1997), which are incorporated by reference. Alternatively, human antibody libraries may be expressed intracellularly and screened via the yeast two-hybrid system. See WO0200729A2, which is incorporated by reference.

Recombinant DNA techniques can be used to produce the recombinant acetylation site-specific antibodies described herein, as well as the chimeric or humanized acetylation site-specific antibodies, or any other genetically-altered antibodies and the fragments or conjugate thereof in any expression systems including both prokaryotic and eukaryotic expression systems, such as bacteria, yeast, insect cells, plant cells, mammalian cells (for example, NS0 cells).

Once produced, the whole antibodies, their dimers, individual light and heavy chains, or other immunoglobulin forms of the present application can be purified according to standard procedures of the art, including ammonium sulfate precipitation, affinity columns, column chromatography, gel electrophoresis and the like. Once purified, partially or to the desired levels of homogeneity, the polypeptides may then be used for performing assay procedures, immunofluorescent staining, and the like.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, acetylated SUMO-1, SUMO-2, SUMO-3 or SUMO-4 protein-binding fragments can be produced through the application of recombinant DNA technology or through synthetic chemistry.

“Humanized” chimeric antibodies can also be used. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

The invention is also directed towards diagnostic methods comprising determining the acetylation state of an acetylation domain within a SUMO protein in a sample of cells from a subject, whereby at least partial acetylation of the acetylated domain of the SUMO protein indicates that cells harboring the SUMO protein are susceptible to induction of cell death. Conversely, detection of unacetylated SUMO proteins (or partial acetylation of SUMO is not detected) indicates that cells harboring the SUMO protein are not susceptible to induction of cell death. Cell death can be apoptotic or it can be cytotoxic. Induction of cell death can be accomplished by any known method of inducing apoptotic or cytotoxic dell death, including but not limited to administration of chemotherapeutic agents discussed herein.

In one embodiment, the acetylation domain that is analyzed in the methods of the invention is an acetylation domain from SUMO-1 (SEQ ID NO:1), SUMO-2 (SEQ ID NO:2), SUMO-3 (SEQ ID NO:3), and/or SUMO-4 (SEQ ID NO:4).

In one embodiment, the acetylation state of the acetylation domain is determined using any of the antibodies or antibody fragments disclosed and described herein.

The diagnostic methods may or may not comprise methods quantifying acetylation at an acetylation domain or other acetylation site described herein. For example, peptides, including AQUA peptides of the invention, and antibodies of the invention are useful in diagnostic and prognostic evaluation of cancer or cancer therapy outcome.

Methods of diagnosis can be performed in vitro using a biological sample, e.g., blood sample, lymph node biopsy, tissue biopsy, including tumor biopsy, normal biopsy, neoplasia biopsy, etc., from a subject, or in vivo. The acetylation state or level at acetylation domains or other acetylation sites described herein may be assessed. At least a partial acetylation of the SUMO protein indicates that cells harboring the SUMO protein are susceptible to induction of cell death via cytotoxic therapeutics and/or apoptotic-inducing compounds. Conversely, detection of unacetylated SUMO protein or lack of detection of acetylated SUMO indicates that cells harboring the SUMO protein are not susceptible to induction of cell death via traditional cytotoxic therapeutics and/or apoptotic-inducing compounds.

In one embodiment, the acetylation state or level at an acetylation domain or other acetylation site is determined by an AQUA peptide comprising the acetylation site. The AQUA peptide may be acetylated or unacetylated at the specified lysine position.

In another embodiment, the acetylation state or level at an acetylation domain or other acetylation site is determined by an antibody or antigen-binding fragment thereof, wherein the antibody specifically binds the acetylation domain or another acetylation site. The antibody may be one that only binds to the acetylation site when the lysine residue is acetylated, but does not bind to the same sequence when the lysine is not acetylated; or vice versa.

In particular embodiments, the antibodies or fragments of the present application are attached to labeling moieties, such as a detectable marker. One or more detectable labels can be attached to the antibodies. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

A radiolabeled antibody in accordance with this disclosure can be used for in vitro diagnostic tests. The specific activity of an antibody or fragment to the ligand depends upon the half-life, the isotopic purity of the radioactive label, and how the label is incorporated into the biological agent. In immunoassay tests, the higher the specific activity, in general, the better the sensitivity. Radioisotopes useful as labels, e.g., for use in diagnostics, include but are not limited to iodine (¹³¹I or ¹²⁵I), indium (¹¹¹In), technetium (⁹⁹Tc), phosphorus (³²P), carbon (¹⁴C), and tritium (³H).

Fluorophore and chromophore labeled biological agents can be prepared from standard moieties known in the art. Since antibodies and other proteins absorb light having wavelengths up to about 310 nm, the fluorescent moieties may be selected to have substantial absorption at wavelengths above 310 nm, such as for example, above 400 nm. A variety of suitable fluorescers and chromophores are described by Stryer, Science, 162:526 (1968) and Brand et al., Annual Review of Biochemistry, 41:843-868 (1972), which are hereby incorporated by reference. The antibodies or fragments can be labeled with fluorescent chromophore groups by conventional procedures such as those disclosed in U.S. Pat. Nos. 3,940,475, 4,289,747, and 4,376,110, which are incorporated by reference.

The control may be parallel samples providing a basis for comparison, for example, biological samples drawn from a healthy subject, or biological samples drawn from healthy tissues of the same subject. Alternatively, the control may be a pre-determined reference or threshold amount. If the subject is being treated with a therapeutic agent, and the progress of the treatment is monitored by detecting the lysine acetylation state level at an acetylation domain or acetylation site of a SUMO protein, a control may be derived from biological samples drawn from the subject prior to, or during the course of the treatment.

In certain embodiments, antibody conjugates of antibody fragment conjugates for diagnostic use in the present application are intended for use in vitro, where the antibody or fragment is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. In certain embodiments, secondary binding ligands are biotin and avidin or streptavidin compounds.

Antibodies of the invention may also be optimized for use in a flow cytometry (FC) assay to determine the activation/acetylation status of a target signaling protein in subjects before, during, and after treatment with a therapeutic agent targeted at inhibiting or promoting lysine acetylation at the acetylation domains or other acetylation sites disclosed herein. For example, bone marrow cells or peripheral blood cells from patients may be analyzed by flow cytometry for target signaling protein acetylation, as well as for markers identifying various hematopoietic cell types. In this manner, aceylation status of the normal and/or malignant cells may be specifically characterized. Flow cytometry may be carried out according to standard methods.

Alternatively, antibodies of the invention may be used in immunohistochemical (IHC) staining to detect differences in signal transduction or protein activity using normal and diseased tissues. IHC may be carried out according to well-known techniques. See, e.g., Antibodies: A Laboratory Manual, supra.

Peptides and antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the methods comprise utilizing two or more antibodies or AQUA peptides of the invention. In one preferred embodiment, two to five antibodies or AQUA peptides of the invention are used. In another preferred embodiment, six to ten antibodies or AQUA peptides of the invention are used, while in another preferred embodiment eleven to twenty antibodies or AQUA peptides of the invention are used.

Acetylation state can also be assessed in other ways and the diagnostic and predictive methods of the present invention are not necessarily limited by the methods of determining acetylation levels. For example, Acetylation can be further quantified with ³H- or ¹⁴C-Acetyl-Coenzyme radioactive labeling, followed by enzymatic digestion and Mass Spectrometry. In this case, standard Multiple Reaction Monitoring (MRM) transitions are quantified using specific Software. The area under MRM peak transitions corresponding to the SUMO non acetylated and acetylated peptides are integrated, and relative ratios between tryptic peptide pairs are determined in samples derived from equal loading standards. For further validation of data, MRM based MIDAS workflow (multiple reaction monitoring initiated detection and sequencing). This procedure can be used to trigger dependent acquisition of product ion scans (MS/MS) using a hybrid quadruple-linear ion trap instrument, e.g., 4000 Q Trap from Applied Biosystems, that confirms the charge state and monoisotopic mass of the potential acetylated peptide and the location of acetylation

In certain embodiments the diagnostic methods of the application may be used in combination with other cancer diagnostic tests.

Other embodiments of the diagnostic methods are methods directed towards predicting the responsiveness of a subject to a cancer treatment, with the predictive methods comprising determining the state of acetylation of an acetylation domain of a small ubiquitin-like modifier protein (SUMO protein) in a sample of the cells obtained from the subject in need of the cancer treatment. At least a partial acetylation of the acetylated domain of the SUMO protein indicates that the subject is more likely than not to respond positively to the cancer treatment. The same methods and compositions, e.g., antibodies, that care used in determining acetylation in the diagnostic methods can be used in the predictive methods for clinical outcome.

Using the methods and composition of the present invention described herein, clinicians are able to determine the acetylation state of SUMO proteins in tissue. A state of at least partial acetylation of SUMO proteins in the tissue or body fluid sample from the subject indicates that the abnormal tissue in the subject would respond to a cancer therapy, i.e., that the cancer therapy would be effective in killing or inhibiting the growth of the abnormal tissue. Conversely, unacetylated SUMO proteins (or partial acetylation of SUMO that is not detected) in the tissue or body fluid sample from the subject would indicate that the abnormal tissue in the subject would not respond to a cancer therapy, i.e., that the cancer therapy would not be effective in killing or inhibiting the growth of the abnormal tissue.

The predictive value of the methods herein need not be absolute. In other words, the predictive methods described herein need only show that it is more likely than not that the cancer therapy would be effective if partial acetylation of SUMO proteins be detected. Similarly, the predictive methods described herein need only show that it is more likely than not that the cancer therapy would not be effective if unacetylated SUMO proteins are detected (or partial acetylation of SUMO is not detected). Moreover, the confidence levels in clinical outcome (responsiveness to therapy) can be but are not necessarily correlative with levels of acetylation of SUMO. In other words, confidence levels of clinical outcome may or may not necessarily increase as levels of detected acetylated SUMO increase. The predictive methods can be repeated on the same subject at different time points to determine changes in state of acetylation of SUMO proteins. Changes over baseline levels may also indicate a change in responsiveness to the cancer therapy. Baseline levels can be established for an individual or can be established by analyzing a population of individuals. Further, baseline levels may be analyzed in normal or abnormal tissue or body fluid.

As used herein and for all embodiments, “abnormal tissue” is cancerous or non-cancerous tissue or cells not present in normal, healthy individuals, e.g., malignant or non-malignant tumors, hyperpasia, neoplasia, cysts and abnormal white blood cells to name a few.

The types of therapy for which the predictive methods may be useful include but are not limited to anti-cell growth therapies. Anti-cell growth therapies include both apoptotic-inducing compounds as well as cytotoxic compounds. One embodiment of anti-cell growth therapy is a cancer therapy. Examples of cancer therapies are well known in the art and include, but are not limited to, alkylating agents, antimetabolites, anthracyclines, plant alkaloids, topoisomerase inhibitors and tyrosine kinase inhibitors. Specific examples of cancer therapies include but are not limited to podophyllotoxin, etoposide, etoposide phosphate, teniposide, cisplatin, carboplatin, oxaliplatin, mechlorethamine, cyclophosphamide, chlorambucil, ifosfamide, purines such as azathioprine and mercaptopurine, pyrimidines, Vincristine, Vinblastine, Vinorelbine, Vindesine, paclitaxel, taxol, docetaxel, irinotecan, topotecan, amsacrine, dactinomycin, doxorubicin, epirubicin, and bleomycin.

Antibodies and peptides (including AQUA peptides) of the invention may also be used within a kit for detecting or quantifying the acetylation state or level of acetylation at an acetylation domain or other acetylation site disclosed herein, comprising at least one of the following: an AQUA peptide comprising the acetylation domain or other acetylation site, or an antibody or antibody fragment that binds to an acetylated amino acid sequence comprising the acetylation domain or other acetylation site of a SUMO protein. Such a kit may further comprise a packaged combination of reagents in predetermined amounts with instructions for performing the diagnostic assay. Where the antibody or fragment is labeled with an enzyme, the kit will include substrates and co-factors required by the enzyme. In addition, other additives may be included such as stabilizers, buffers and the like. The relative amounts of the various reagents may be varied widely to provide for concentrations in solution of the reagents that substantially optimize the sensitivity of the assay. Particularly, the reagents may be provided as dry powders, usually lyophilized, including excipients that, on dissolution, will provide a reagent solution having the appropriate concentration.

Any of the methods or compositions of the present invention can be performed in virtually any setting, such as an in vivo, ex vivo, in situ or in vitro setting. For example, methods of diagnosis or methods of predicting responsiveness may be performed in cell culture, or may be performed in an intact organism. Moreover, any combination of any two or more of any of the embodiments described herein are contemplated.

The following examples are illustrative and are not intended to limit the scope of the invention described herein.

### Examples

### Methods

Purified SUMO-1, SUMO-2 and SUMO-3 were purchased from Biomol. (#UW9190, UW9200, UW9210, respectively). CBP protein was also from Biomol. The anti-SUMO antibodies were from Zymed (monoclonal); or Santa Cruz (monoclonal D11). The anti-Flag antibodies were from SIGMA Aldrich (M2 monoclonal; anti-rabbit polyclonal). The p53 antibody employed for chromatin immuno-precipitation assays was from Santa Cruz (FL393). A polyclonal antibody was raised against peptide sequences SEIHF{K-Ac}VKMTTHLKK (37-1Ab) and purified by double affinity column. The peptide was N-acetylated and a cysteine was added at the C-terminus to improve immunogenicity. Unless otherwise stated, the experiments described herein were conducted with the 37-1Ab. This first antibody (37-1Ab) had a titer of from about 1:3000 to about 1:6000 as assessed with a standard ELISA test. Another polyclonal antibody was raised against peptide sequences DSSEIHF{K-Ac}VKMTTHLKKLKES (37-2Ab) and purified by double affinity column. This second peptide was N-acetylated and a cysteine was added at the C-terminus to improve immunogenicity. This second antibody (37-2Ab) had a titer of about 1:512,000 as assessed with a standard ELISA test.

The p53-SUMO-1 chimerical proteins (p53-SUMO and p53-ΔGG) and the corresponding lysine mutants were produced by amplifying SUMO-1 domain from an existing plasmid expressing full length SUMO-1 by PCR followed by site directed mutagenesis. The PCR products were cloned into the pcDNA4/TO-Flag-p53 vector whose construction has been previously described. For the generation of non-conjugated SUMO-1, an N-terminal Flag epitope was added to SUMO-1 by PCR amplification of SUMO-1 using the following primer: N-term Flag-SUMO, 5′AAAGGATCCGCCATGGATTATAAAGATGATGATGATAAGGGATCTGACCAGGAGGCA-3′. The resulting PCR product was then cloned into pcDNA4/TO. Mutagenesis of the lysine residues in SUMO-1 was conducted with the ExSite PCR-Based Site-Directed Mutagenesis Kit (Stratagene).

Acetylation Assays.

Acetylation reactions were typically assembled in a 30 μl volume, containing HAT buffer (1×25 mM Tris-HCl ph 7.9; 50 mM NaCl, 1 mM DTT), 6 μl of ¹⁴C-Acetyl-Coenzyme A (Amersham) or cold Acetyl-Coenzyme A (lithium salt, from Roche), 1-2 μg CBP, 0.5-1 μg of SUMO proteins.

Cell Lines, Transfections, and Generation of the H1299 Tetracycline Inducible Cells.

Tetracycline inducible cell lines were generated with the T-REx system (Invitrogen), as described previously. After selection with proper antibiotics, polyclonal cell cultures were isolated and first examined for p53-expression levels by employing both immuno-fluorescence and immuno-blot assays. Typically three-to-five clones showing homogenous levels of expression (i.e., more than 80% of cells expressing p53 proteins) were pooled together and used for further studies.

Cell Cycle Analysis.

Flow cytometry was performed as described previously by Knights, C. D., et al. J. Cell Biol. 173(4):533-544 (2006).

Immuno-Blots, Immuno-Precipitations and Treatments.

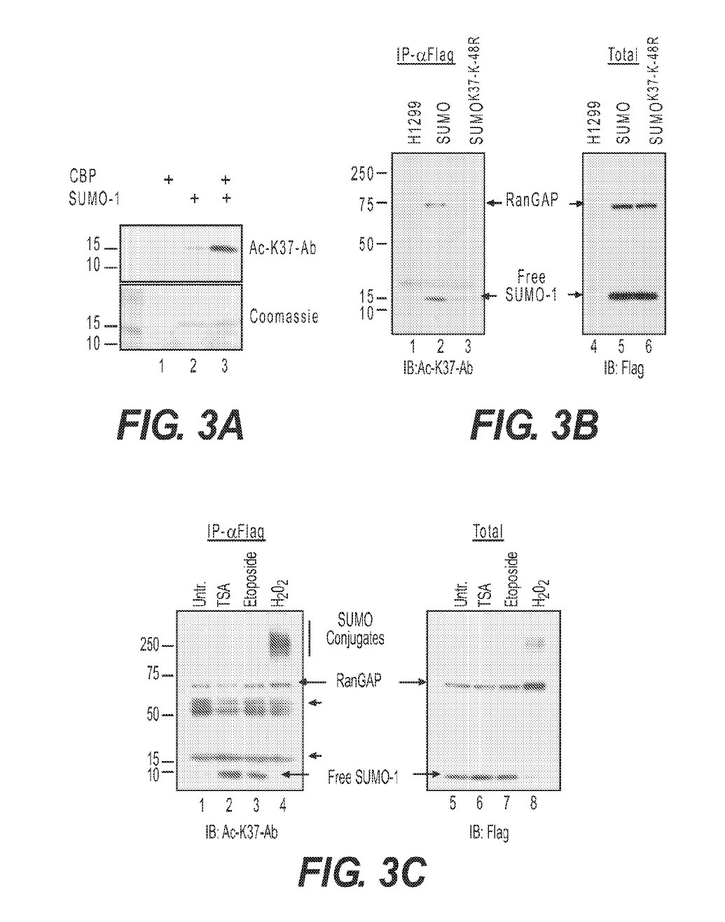

Unless otherwise indicated, cell extracts were prepared in RIPA buffer (0.1% SDS; 50 mM Tris.Cl, pH 6.8; 150 mM NaCl; 0.5% Nonidet P-40; 2 mM EDTA) supplemented with protease a protease inhibitor mixture, with TSA (500 μM) and with 10 nM NEM as an inhibitor of de-sumoylation. Treatment with etoposide was performed at 100 μM overnight. When probed with the anti-SUMO or anti-acetyl-K37 antibody (37-1Ab) in direct immuno-blot, membranes where treated with 6 M Guanidine for 30 minutes, followed by incubation in blocking solution. The Ac-K37 (37-1Ab) antibody was used to a 1:500 dilution in direct immuno-blots on in vitro acetylation reactions (i.e., FIG. 3A) and to a 1:250 dilution for the experiments in cells and in murine tumors. In experiments using tumor samples from animals, PDF membranes were denatured in 6M guanidine HCl, then re-natured, and subjected to immuno-blot analysis with the anti-SUMO or the anti Ac-K37 (37-1Ab) specific antibodies. For H₂O₂ treatment cells were first trypsinized and collected in 10 ml of growth media. H₂O₂ was added drop by drop at a final concentration of 100 μM. Cells were then incubated at 37° C. for 20 minutes, washed twice with PBS and lysed in RIPA buffer.

Purification of SUMO-1 for Tryptic Digestion and Mass Spectrometric Analysis.

The entire purification reaction from the Flag IP from cells was used, or the soluble in vitro reaction derived from the CREB binding protein (CBP)-directed acetylation. In the former case, 50 dishes of H1299 (15 cm) cells were grown to confluence, treated with TSA (500 μM) and induced with tetracycline for 24-36 hours. Cell extracts were prepared in RIPA buffer complemented with inhibitors as described previously, pre-absorbed on agarose beads, and then subjected to immunoprecipitation with 1.5 ml of anti-Flag-M2 immuno-affinity column, overnight. Beads were washed 7 times in 10 volumes of RIPA buffer, then for one hour with 15 volumes or RIPA buffer. Immuno-precipitates were subjected to a final wash with buffer containing 25 mM Hepes pH 7.5; 100 mM KCl; 10% Glycerol, 0.4 mM EDTA, 10 mM BME), and eluted in the same buffer containing the flag peptide at 400 ug/ml. Three subsequent elutions in 0.5 (v/v) of elution buffer were carried out and pulled together. For mass spectrometry analysis, samples were dried under vacuum and resuspended in 25 mM ammonia bicarbonate, reduced by incubation at 60° C. for 1 hr with 5 mM TCEP (2, carboxy ethyl phosphine) and alkylated with 10 mM MMTS (methyl methane thio sulfonate) for 10 minutes at room temperature. 1 μg of sequencing grade trypsin was used for digestion (Promega, USA) that was carried out at 37° C. for 18 hrs. Following desalting with C18 reverse phase spin columns (Nest group, USA) the eluted polypeptides were dried and resuspended in 20 μl of solvent A (98% water, 2% acetonitrile and 0.1% formic add) and finally analyzed with nanoLCMS/MS or nanoLC MRMMS/MS.

Micro-Array Analysis.

Cells were harvested for RNA extraction and 7 μg of total RNA was used for cDNA and biotinylated cRNA synthesis. Expression profiling analysis was performed using the HG-U133A 2.0 human Affymetrix high-density oligonucleotide micro-array. Each gene-chip was used for a single hybridization with RNA isolated from one cell line. Each sample was run in a duplicate or in triplicate. Two normalization processes were used: one for chip-chip comparisons (scaling factors), and one for gene-gene comparison (normalization to the average of the naïve signal intensities for each gene). The scaling factor determinations were done using default Affymetrix algorithms (MAS 5) with a target intensity of chip sector fluorescence to 800. The use of Affymetrix MAS 5.0 signal intensity values, together with a “present call” noise filter achieves an excellent signal/noise balance relative to other probe set analysis methods (dchip, RMA). Data analyses were limited to probe sets that showed 1 or more “present” (P “calls”) in the 8 genechip profiles in our complete dataset. Data were analyzed using the GeneSpring software (Silicon Genetics).

Chromatin Immunoprecipitation Assays.

ChIP assays were performed as is known in the art. Briefly, 2×107 H1299, or H1299 expressing p53, p53-SUMO, or p53-SUMOK45R cells were grown in the absence or presence of tetracycline and subsequently exposed to 1% formaldehyde-PBS solution for 13 min at room temperature. The extracts were sonicated after lysis to obtain DNA fragments of lengths comprised between 300-800 bp. Chromatin solutions were precipitated overnight with rotation using a rabbit polyclonal anti-p53 antibody (FL393). On the following day, protein A agarose beads that had been previously blocked with salmon sperm DNA and BSA were added to each reaction to precipitate DNA-p53 complexes. These were washed and then incubated at 65° C. overnight in parallel with ‘input’ samples to reverse the cross linking. DNA was isolated with the Qiagen-PCR purification kit. The precipitated DNA was then subjected to PCR reactions for 30-35 cycles. Quantification of ChIP assays was performed as follows. The mean relative intensity of input and experimental ChIP PCR bands was determined using the histogram feature of Adobe Photoshop 7.0. Input intensities were normalized to correct for minor differences in the total amount of DNA contained in the samples. Experimental band intensities were then adjusted against their corresponding normalized input signal. Fold change represents the comparison of normalized p53 or p53-SUMO band intensity compared to normalized H1299 band intensity.

Quantitative Real Time PCR (qRT-PCR).

Parental H1299 cells or H1299 cells expressing p53, p53-SUMO or Flag-SUMO were treated with tetracycline for 48 hrs followed by extraction of total RNA. Reverse transcriptase PCR (SuperScript III first-strand synthesis system, Invitrogen) was performed with random hexamer primers and 2 mg of RNA to obtain sufficient cDNA for analysis. cDNA samples were then mixed with gene specific primers (generated by SciEd Central, Scientific & Educational Software) and iQ SYBR green super mix (BioRad) according to the manufacturer's instructions. Samples were then analyzed using an ICycler iQthermocycler (BioRad) and software version 3.1.7. Each cDNA was analyzed in duplicate. Cycle counts were normalized against b-actin and reported relative to H1299 cells using the DDCT method.

Mouse Models.

Mice carrying a transgene composed of the mouse mammary tumor virus-long terminal repeat (MMTV-LTR) linked to sequences encoding the tetracycline responsive reverse transactivator (tTA) for “tet-off” gene regulation and a transgene composed of the tetracycline operator (tet-op) promoter linked to sequences encoding the Simian Virus 40 T Antigen (TAg) on a C57Bl/6 background (Ewald et al; Tilli et al.) were maintained on regular mouse chow and euthanized in accordance with institutional and federal guidelines approved by the Georgetown University Animal Care and Use Committee. Submandibular salivary gland preneoplastic and tumor tissue was isolated at the time of necropsy. One half was formalin fixed and processed for Hematoxylin and eosin section staining; the other half was flash frozen and stored at −20° C. until used for biochemical experiments.

Results

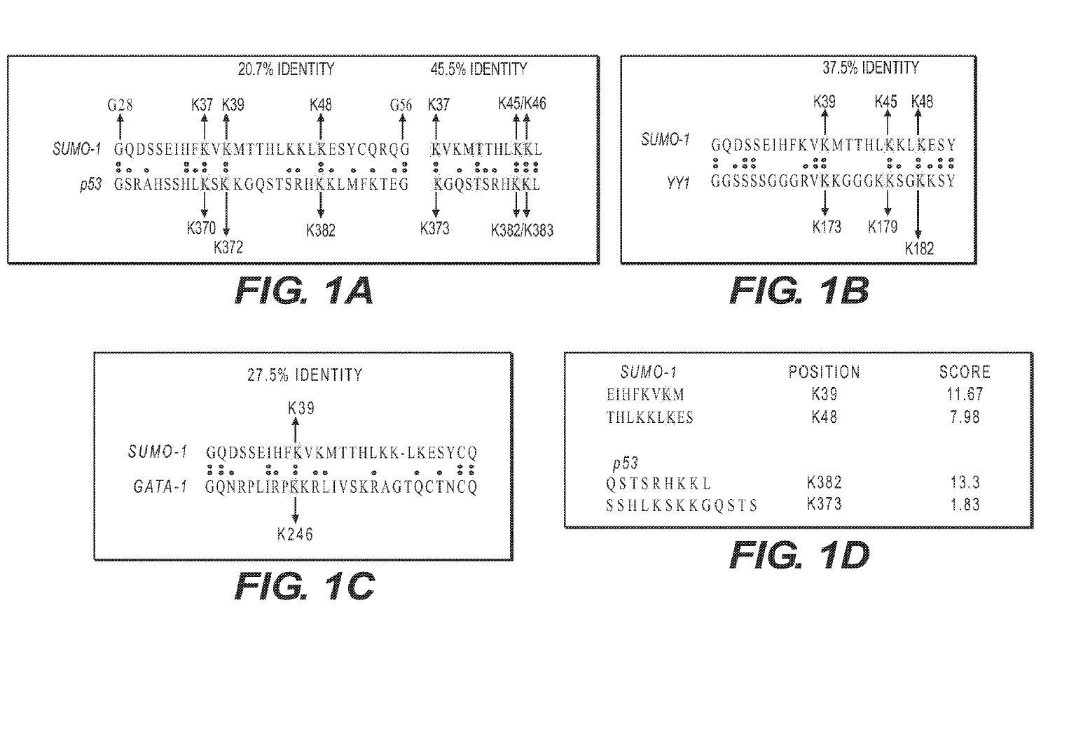

K37-K39 and K45-K48 of SUMO-1 aligned with p53 lysines targeted for acetylation by p300/CBP at position K370, K372, K373 and K382-K383 (FIG. 1A, in red and indicated by arrows). In both SUMO-1 and p53 these lysines are surrounded by similarly charged amino acid residues, a characteristic that is important for substrate recognition by acetylases. This SUMO-1 region aligned with known acetylation sites of other proteins as well, such as YY1, GATA-1 and FEN-1 (FIG. 1 B,C).

The discovery of such similarities implied that this SUMO-1 domain might functionally mimic the acetylated state of similar domains of transcription factors. Two approaches were used to determine if the identified SUMO-1 domain was acetylated. First, acetylation assays were performed in vitro using purified SUMO-1 protein and the catalytic domain of several acetyltransferases, specifically of CBP and PCAF, in the presence of 14C-acetyl CoA. As shown in FIG. 2A purified SUMO-1 was efficiently acetylated by CBP (lane 3), but not by PCAF (not shown). To preliminarily map the site(s) of acetylation, we tested the ability of cold synthetic polypeptides corresponding to specific SUMO-1 regions, to inhibit CBP-mediated acetylation of the full-length protein in a reaction containing 14C-acetyl CoA. Two polypeptides comprising lysines at position K37-K39, and K45-K46-K48 completely blocked acetylation (FIG. 2B, lanes 3 and 4), suggesting that these residues are responsible for the majority of the SUMO-1 acetylation signal. By contrast a peptide containing lysines at position K25 and K26 only weakly abrogated CBP-mediated acetylation of full length SUMO-1 (FIG. 2B, lane 2).

Second, to assess whether acetylation occurs in vivo, an epitope Flag-tagged-SUMO-1 was expressed in the human H1299 lung cancer cell line under the control of a tetracycline-regulated promoter. In this system the expression of exogenous SUMO-1 leads to a compensatory down-regulation of the endogenous protein, therefore counterbalancing over-expression. Following treatment of cells with the class I/II deacetylase inhibitor TSA, SUMO-1 complexes were purified and the total elution material containing both free non-conjugated SUMO and sumoylated proteins, was digested with trypsin and analyzed using nano-LC/MS/MS and Multiple Reaction Monitoring (MRM) detection. These purification experiments enriched for SUMO-1 in a specific fashion. To determine whether the same residues are acetylated in vitro and in cells, mass spectrometry was simultaneously performed on the CBP-directed in vitro acetylation reactions as well. Although it is often assumed that trypsin does not cleave N-acetylated lysine residues, it has been previously observed that tryptic digestion can result in polypeptides containing C-terminal acetylated lysine residues. Consistent with this, in cells we detected well represented MRM transitions corresponding to tryptic polypeptides 26-VIGQDSSEIHFK(Ac)-37 and 26-VIGQDSSEIHFK(Ac)VK-39 and to their non acetylated forms, that undoubtedly identified acetylation of K37. To obtain an approximate determination of the stoichiometry of acetylation at this residue, the relative MRM signals were compared between acetylated and non-acetylated polypeptides. This analysis revealed a rough relative abundance of approximately 42% of K37 acetylation in the total amount of purified SUMO-1. Additional polypeptides were detected that carried individual acetylation events at K39, as well as combination of acetylation at both K37 and K39 and at K45, K46 and K48. Thus, a significant fraction of SUMO-1 is acetylated at these two residues in vivo, although additional experiments are certainly needed to determine the absolute concentration of the acetylated polypeptides in cells. Less represented post-translational modifications were also identified, consisting of acetylation and palmitoylation of K24, N-terminal acetylation, and acetylation of K45, K46 and K48.

From the mass spectrometric analysis performed on the in vitro reactions, it was determined that CBP was able to acetylate the same residues that were found acetylated in vivo, specifically K37, K39 and K48. It is significant that these SUMO-1 lysine residues are conserved among different family members (FIG. 2C) in spite of the only modest overall homology between SUMO-1 and SUMO-2/3 (approximately 42%). Residues surrounding K37-K39 are also well conserved, possibly identifying an acetylation motif in all SUMO proteins. In agreement with this speculation, SUMO-2 and SUMO-3 were also robustly acetylated by CBP (FIG. 2D, lanes 6-7). As such, these results unexpectedly revealed that SUMO-1 is modified by acetylation at multiple residues, both in vitro and in cells.

To study how acetylation influences the activity of SUMO-1, a polyclonal antibody was raised against a peptide acetylated at K37 (Ac-K37-Ab). The specificity of this antibody was first assessed in in vitro acetylation assays (FIG. 3A). A robust signal was seen in the Ac-K37-Ab immuno-blot only in the presence of an acetylation reaction (compare lane 3 with lane 2). As a second approach, a SUMO-1 mutant was constructed where the acetylated lysines K37-K39 and K45, K46 and K48 were substituted with the non-acetylable, charge preserving amino acid arginine residues (SUMO-1K37-48R). In the experiments shown in FIG. 3B cell extracts derived from control H1299 cells or from cells expressing SUMO-1 or SUMO-1K37-48R were first immuno-precipitated with the anti-Flag antibody and then probed in immuno-blot with the Ac-K37-Ab (left panel). Meanwhile the analysis of the total SUMO-1 and SUMO-1K37-48R-derived extracts with the anti-Flag antibody showed that SUMO-1 exists in cells mostly as a nonconjugated, free pool, and as conjugated to RanGAP (FIG. 3B, lanes 5 and 6), as also noted by others. Significantly, the Ac-K37-Ab reacted with both the free- and RanGap-bound fraction of SUMO-1, but not of SUMO-1K37-48R (FIG. 3B, compare lane 2 with lane 3), further demonstrating specificity of this antibody.

SUMO-1 expressing cells were treated with either TSA or with the DNA damaging agent etoposide. As shown in FIG. 3C, an acetylation signal stronger than that seen in untreated cells was detected again on free SUMO-1, in both TSA and etoposide treated cells (FIG. 3C, compare lanes 2 and 3 with lane 1), while the levels of acetylation seen on RanGAP were only modestly influenced by these treatments. To determine whether SUMO-1 is acetylated when conjugated to substrates other than RanGAP, cells were treated with Hydrogen Peroxide (H₂O₂,), which produces an accumulation of SUMO conjugates due to inactivation of SUMO peptidases. In these conditions the Ac-K37-Ab reacted directly with several high molecular weight SUMO-1 substrates (FIG. 3C, lane 4). From the combination of these experiments it appears that SUMO-1 can be acetylated when bound to at least a set of its targets.

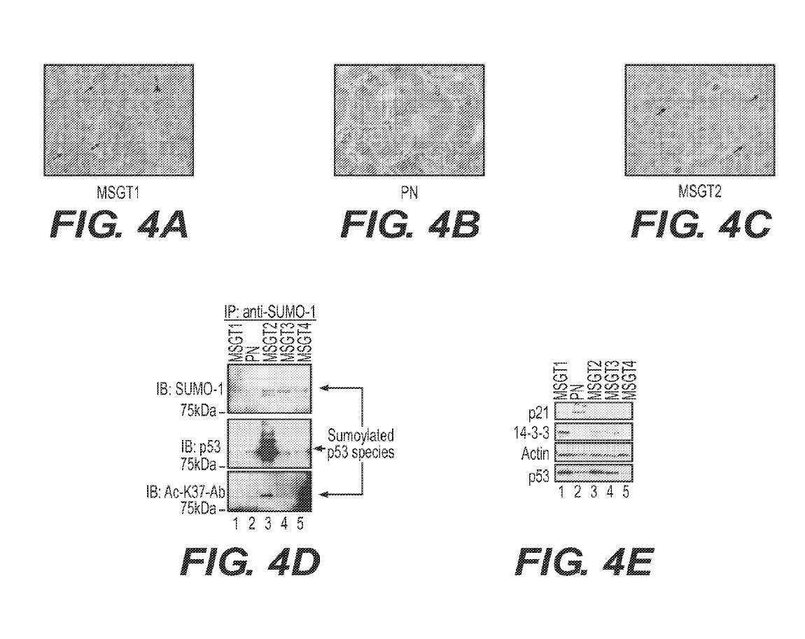

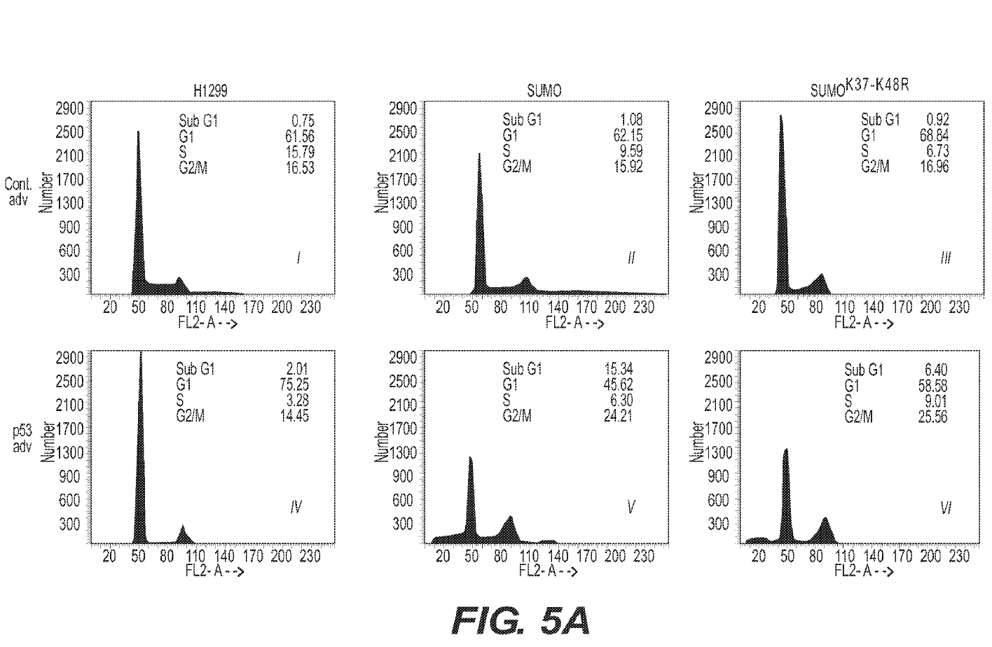

The p53 tumor suppressor is an important SUMO-1 target. Although p53 sumoylation has been detected in cells as a consequence of SUMO-1 over-expression, whether this modification occurs in tumors has not been explored yet. Transgenic animals expressing SV40 large Tag develop ductal hyperplasia in the sub-mandibular gland at around four months of age that eventually progresses to adenocarcinoma within the first year. Loss of p53 accelerates the onset of adenocarcinomas demonstrating that p53 acts as a barrier to tumor progression in these animals. Sub-mandibular tissue was excised and examined from animals with suspected preneoplasia but no palpable or endured tumor on one side (PN), and one palpable tumor on the contra lateral side (MSGT1), as well as frankly malignant lesions of three other animals (MSGT2-to-4). Prototypical histological sections of these samples are shown in FIG. 4A-C. Cell extracts from these tissues were prepared, and subjected to analysis with anti-SUMO-1-, anti-p53-, and anti Ac-K37-Ab antibodies. With this analysis it was determined that sumoylated forms of p53 with different molecular weight, i.e., higher than the expected normal size, were detected in all these tissues (FIG. 4D, indicated by arrows). These species reacted with both the anti-SUMO and anti-p53 antibody. In the tumors where the highest levels of p53 sumoylation were detected, reactivity with the Ac-K37-Ab antibody was also seen (FIG. 4D, lanes 3 and 4). Thus, in some mouse tumors, SUMO-1 is acetylated when conjugated to p53. It was also found that p21 levels were higher in PN relatively to frankly malignant lesions, correlating with higher levels of p53 sumoylation seen in these latter, while the contrary was true for 14-3-3 sigma (FIG. 4E, compare lane 2 with lane 3 and 4).

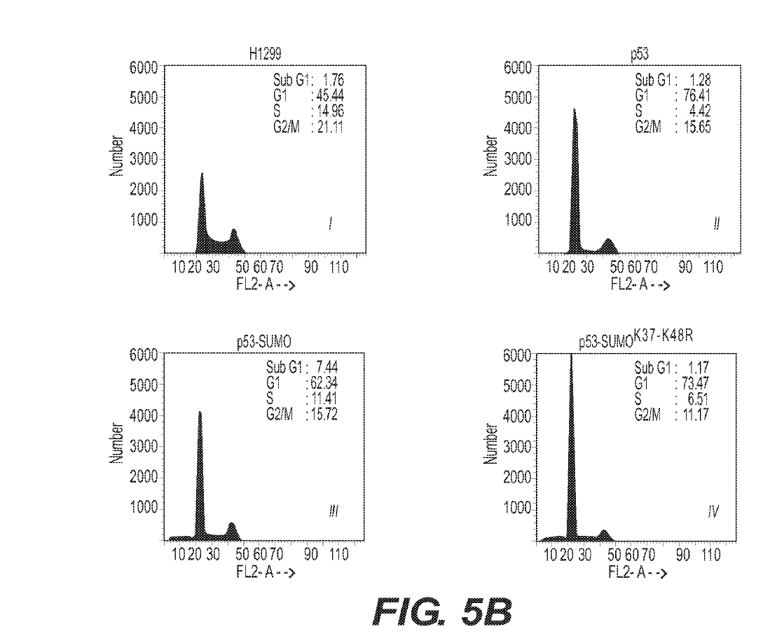

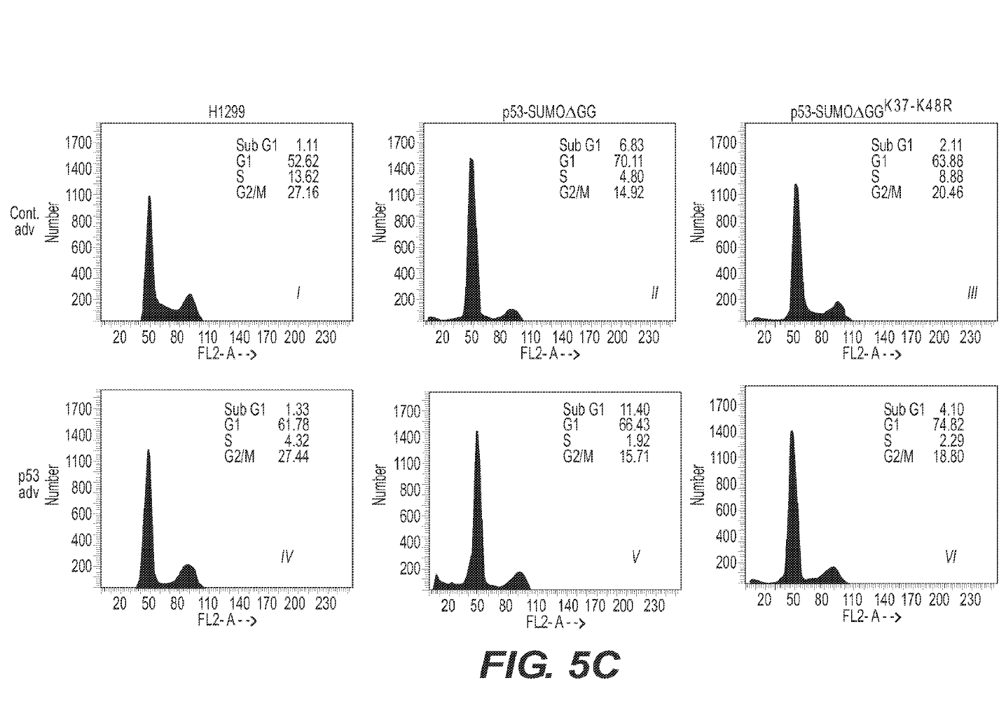

To study how p53 sumoylation influences proliferation, several strategies were employed. First, p53 was expressed in either naïve H1299 cells, or in the H1299 cell line harboring SUMO-1 or SUMO-1K37-48R. To achieve a homogenous level of p53 expression, a replication deficient adenovirus was used as is well known in the art. As shown in FIG. 5A, expression of p53 alone led to an arrest of the cycle at the G1 phase in the H1299 cell line (compare panel i with panel iv). By contrast, 15% of SUMO-1 expressing cells underwent apoptosis in the presence of p53—but not of control adenovirus (panel v versus panel and this effect was partially reverted by SUMO-1K37-48R whose expression resulted in more cells arrested in G1 (panel vi). Similarly to SUMO-1K37-48R, only 2% of apoptotic cells were seen when p53 was expressed in the parental H1299 cell line. This result suggests that sumoylation enhances the apoptotic activity of p53 and underscores the importance of lysines within SAD in this respect.

Chimerical proteins were constructed where one moiety of SUMO-1 was attached as linear fusion to the C-terminal tail of p53. This approach generates a gain of function, stable sumoylated form of p53, which allows the study of this modification in the absence of other unpredictable events. Of similar importance, since sumoylated p53 is present in lower abundance relatively to the non-sumoylated fraction, the use of these chimeric proteins permitted a definition of the biological activities of this underrepresented p53 population. Two chimeras were used for these experiments, one where the last two glycine residues of SUMO-1 required for conjugation were eliminated (p53-SUMOΔGG), and a second one where these residues were left intact (p53-SUMO). To study the activity of the acetylated domain of SUMO-1, SAD, lysine K37-to-K48 were replaced with amino acid residues that destroy acetylation, arginine or alanine, to generate p53-SUMOK37-K48R or p53-SUMOΔGG K37-K48A. In the case of androgen receptor acetylation, alanine and arginine mutants behave identically. p53 proteins were expressed with the tetracycline inducible system in the p53 null H1299 cell line, that has been extensively used for studying p53 signaling. Unlike native p53 whose expression results in a diffuse nuclear staining, p53-SUMO and p53-SUMOΔGG localized in promyelocytic leukemia (PML) nuclear bodies, where p53 was shown to co-localize together with PML in several conditions. In addition, SUMO-1 was found to be acetylated in both p53-SUMO and p53-SUMOΔGG chimeras, indicating that p53-SUMO chimeric proteins recapitulate physiological aspects of p53 sumoylation.

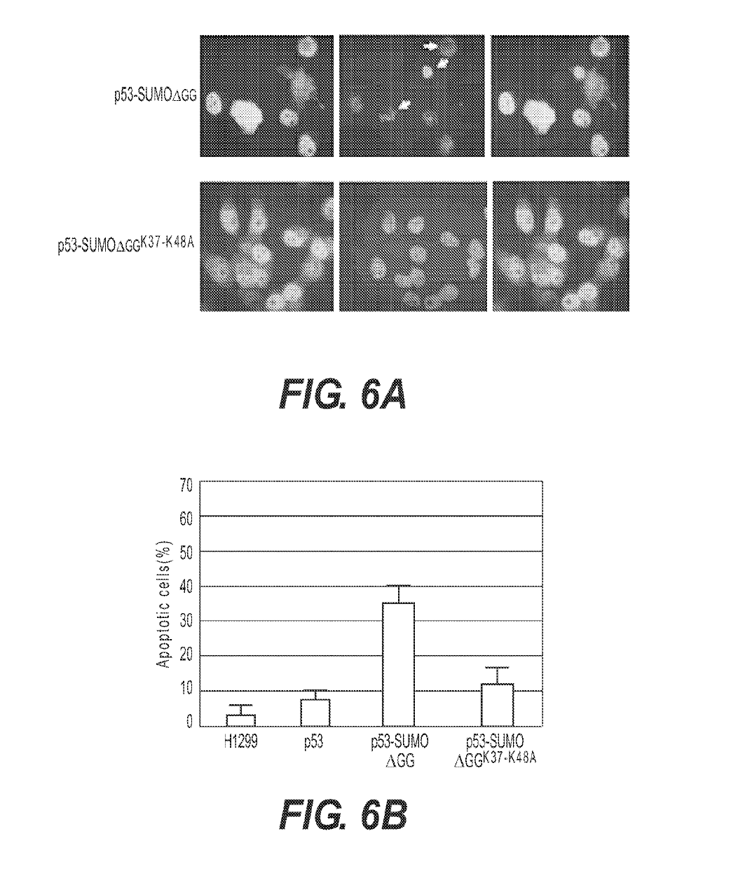

The cell cycle distribution of these cells was next studied and compared to the H1299 cell line expressing naïve p53. A higher percentage of apoptotic cells was again seen in p53-SUMO expressing cells relatively to those harboring either naïve p53 or p53-SUMOK37-K48R, that arrested predominantly in G1 (FIG. 5B, compare panels ii and iii with panel iv). Because sumoylated p53 co-exists with the non-sumoylated fraction, co-expression of how these two populations affects proliferation was examined. Expression of p53 with the adenoviral vector in cells harboring p53-SUMOΔGG (or in p53-SUMO cells) only modestly enhanced apoptosis relatively to cells expressing p53-SUMOΔGG alone (FIG. 5C, compare panels iv and v), while cells harboring p53-SUMOΔGG K37-K48A arrested in G1 in the absence or in the presence of native p53 (compare panels iii and vi with panel iv). Inspection of cells expressing p53-SUMOΔGG and p53-SUMOΔGG K37-K48A via immunofluorescence, revealed the presence of apoptotic fragmented nuclei in the former but not in the latter (FIG. 6A and quantified in FIG. 6B).

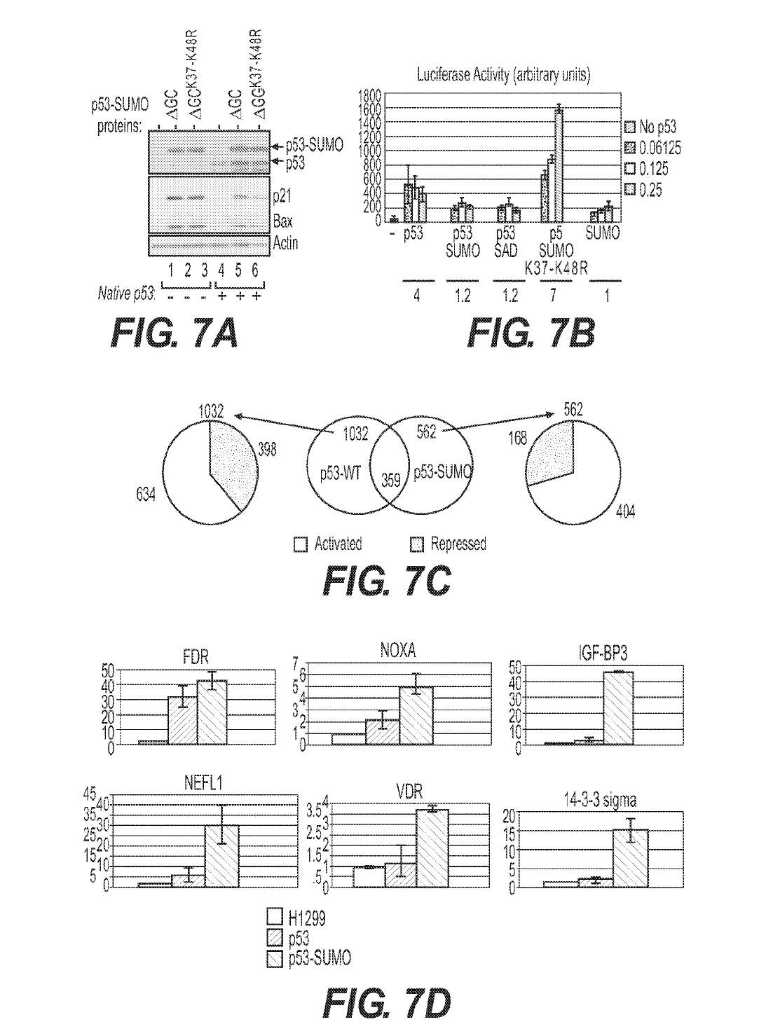

The expression patterns of two of the downstream transcriptional targets of p53, bax and p21, were examined. p21 was induced by naïve p53 and by p53-SUMOΔGG K37-K48A, but not by p53-SUMOΔGG (FIG. 7A, compare lane 4 and 3, with lane 2). A clearly different pattern of expression was instead observed in the case of bax, which was strongly expressed in cells harboring p53-SUMO proteins relative to cells expressing naïve p53 (compare lanes 2-3 with lane 4). Noticeably, the pattern of expression of p21 or bax was unaffected by co-expression of naïve p53 together with p53-SUMO proteins (FIG. 7A, compare lanes 5 and 6 with lanes 2 and 3). First, even though it has been shown that in vitro p53-SUMO can form tetramers with non-sumoylated p53, it seems likely that the presence of non-sumoylated p53 within these tetramers would not significantly modify the ability of sumoylated p53 to influence transcription. Second, the data suggest that lysine residues within SAD might influence the modality by which p53 selects activation of its downstream target genes. Third, these lysine residues also appear essential for p53-mediated induction of apoptosis but not of cell cycle arrest.

A canonical reporter assay was performed by employing a p21-regulated promoter placed upstream of the luciferase gene. To exclude the possibility that observed difference(s), if any, might be due to variations in the levels of the various p53 protein, titration experiments were performed, while expression of non conjugated SUMO-1 provided a control for possible transcriptional effects of SUMO, independently of p53. As shown in FIG. 7B, in cells expressing nonconjugated SUMO-1 a modest induction of p21 reporter activity was seen, therefore luciferase levels detected in these samples were used for normalization. When compared to the background activity of SUMO-1, p53-SUMO was unable to transactivate the p21 reporter relatively to unmodified p53. This result is in full agreement with previous studies showing that purified sumoylated p53 does not support transcription of the p21/WAF promoter in in vitro assays. We further determined that a p53-SUMO chimera lacking part of the N-terminus and the entire C-terminal half of SUMO-1, but containing only amino acids 14 to 55 (p53-SAD) behaved identically to p53-SUMO in that it was similarly unable to robustly stimulate transcription. Thus, the presence of SAD is entirely sufficient to convey the inhibitory effect of SUMO-1 on p21 transcription. Even more significantly, the non-acetylable form of SUMO, p53-SUMOK37-K48R reverted this inhibition and stimulated the p21 reporter. These data imply that reversible acetylation of SADs might switch the activity of SUMO-1 from an inhibitor to an activator of transcription, at least on the p21 promoter.

Due to significant variations of genomic p53 DNA-consensus sites along with differences in chromatin density and architecture, the promoter context is a key determinant of the ability of p53 to select its downstream targets. Gene expression micro-array analysis was performed on cells expressing native p53 or p53-SUMO. The gene expression profiles derived from these cells were compared to those obtained in the parental H1299 cell line and in H1299 cells expressing SUMO-1, employed as controls. Somewhat surprisingly, it was found that very few genes were affected by the expression of SUMO-1 alone. Arrays obtained in H1299 cells as the background were used for comparison. A partial list of genes modulated by p53-SUMO is provided in Table 1.

TABLE 1
Genes Influenced by Native p53 and p53SUMO

| | | p53 | | p53SUMO | |
|---|---|---|---|---|---|
| Name | Symbol | Fold change | p-value | Fold change | p-value |
| neurofilament, light polypeptide 68 kDa | NEFL | 3.68 | 0.025 | 45.41 | 0.016 |
| cytoplasmic FMR1 interacting protein 2 | CYFIP2 | 39.57 | 0.012 | 31.76 | 0.026 |
| growth differentiation factor 15 * | GDF15 | 204.64 | 0.005 | 26.45 | 0.006 |
| ferredoxin reductase * | FDXR | 22.41 | 0.011 | 19.79 | 0.002 |
| membrane-type 1 matrix metalloproteinase | MTCBP-1 | 2.46 | 0.009 | 18.26 | 0.006 |
| cytoplasmic tail binding protein-1 | | | | | |
| tumor protein p53 inducible protein 3 * | TP53I3 | 42.58 | 0.004 | 16.90 | 0.005 |
| insulin-like growth factor binding | IGFBP3 | −1.46 | 0.023 | 15.43 | 0.041 |
| protein 3 * | | | | | |
| stratifin (2) * | SFN (14-3-3σ) | 4.16 | 0.041 | 14.49 | 0.013 |
| cyclin-dependent kinase inhibitor | CDKN1A (Cip1) | 28.00 | 0.008 | 13.23 | 0.018 |
| 1A (p21) * | | | | | |
| polo-like kinase 3 (Drosophila) | PLK3 | 11.08 | 0.035 | 12.53 | 0.027 |
| Mdm2, transformed 3T3 cell double | MDM2 | 18.61 | 0.002 | 11.18 | 0.033 |
| minute 2 (2) * | | | | | |
| Carboxypeptidase M | CPM | 24.10 | 0.011 | 10.04 | 0.008 |
| protein phosphatase 1D magnesium- | PPM1D (Wip1) | 4.23 | 0.004 | 7.03 | 0.014 |
| dependent, delta isoform * | | | | | |
| DEAD (Asp-Glu-Ala-Asp) box | DDX43 | −1.01 | 0.029 | 7.00 | 0.023 |
| polypeptide 43 | | | | | |
| Ras-related associated with diabetes | RRAD | 15.15 | 0.019 | 6.84 | 0.005 |
| BCL2-associated X protein * | BAX | 5.99 | 0.001 | 5.54 | 0.039 |
| phorbol-12-myristate-13-acetate- | PMAIP1 (Noxa) | 1.59 | 0.078 | 5.22 | 0.036 |
| induced protein 1 (2) * | | | | | |
| apoptotic peptidase activating factor * | APAF1 | 1.84 | 0.044 | 4.17 | 0.048 |
| vitamin D (1,25-dihydroxyvitamin D3) | VDR | 1.87 | 0.042 | 3.86 | 0.026 |
| receptor (2) * | | | | | |
| p53 target zinc finger protein * | WIG1 | 6.58 | 0.015 | 3.46 | 0.016 |
| p53-inducible cell-survival factor * | P53CSV | 3.57 | 0.015 | 3.13 | 0.036 |

A schematic diagram of the total number of genes regulated by p53 and p53-SUMO is shown in FIG. 7C. Through this approach it was determined that SUMO-1 may lead to global attenuation of the transcriptional activity of p53. In fact, 1032 genes were found modified by p53, while only 562 were influenced by p53-SUMO. Such reduction occurred predominantly at the expenses of the repressed genes: in fact, native p53 repressed 634 transcripts, while only 168 were inhibited by p53-SUMO, suggesting that, like in the case of other proteins, SUMO-1 alleviates trans-repression. Further, while the global number of activated transcripts was essentially comparable between the two proteins, several genes normally up-regulated by p53 were activated much less efficiently by p53-SUMO, (most noticeably p21) and viceversa, 162 genes were found up-regulated by p53-SUMO relatively to native p53. By taking into consideration only well known and validated p53 targets, it was determined that FDXR, NOXA, IGFBP3, VDR and 14-3-3-sigma were all up-regulated in p53-SUMO-1 expressing cells relatively to p53, while other transcripts such as MDM2, PUMA or AIP1 were under-represented (Table 1). As shown in FIG. 7D, real time PCR confirmed modulation of some of these transcripts in p53-SUMO expressing cells.

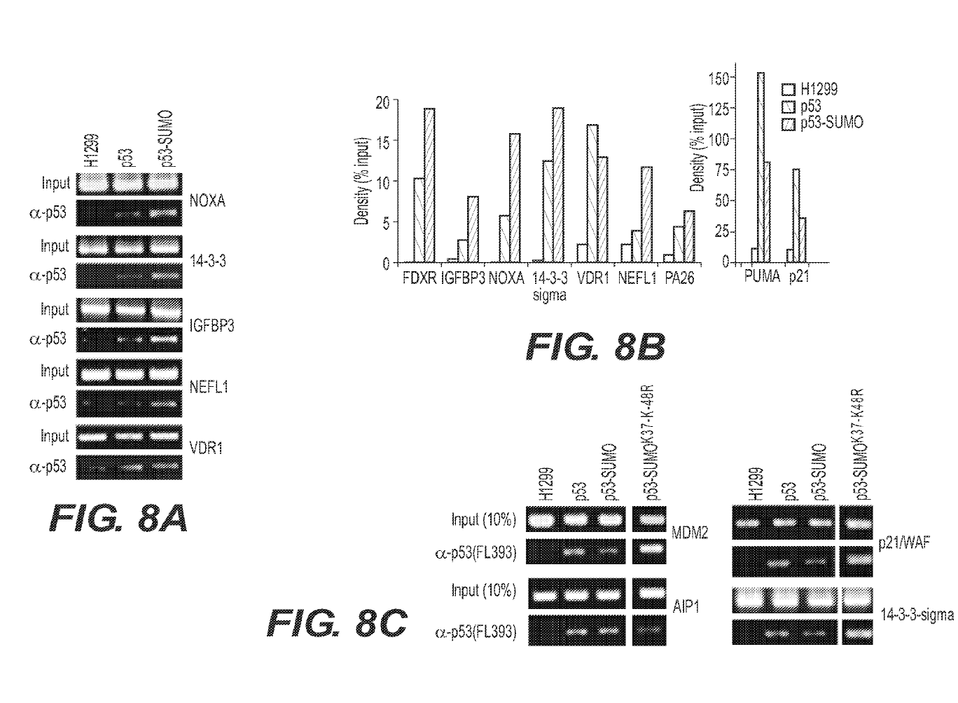

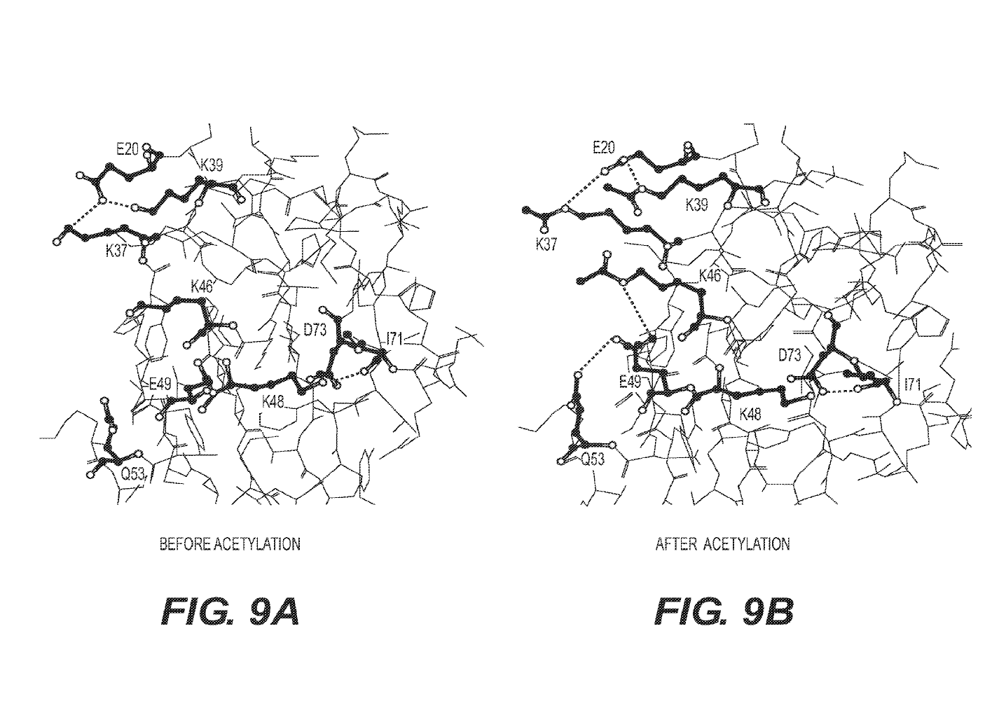

These data suggest that SUMO-1 restricts the transcriptional activity of p53 by alleviating trans-repression, while stimulating the transactivation potential on a discrete number of genes. Chromatin immuno-precipitation assays (ChIP) was performed on a variety of p53-regulated genes that, based on the micro-array assays, showed similar or differential modulation by p53-SUMO compared to native p53 (FIG. 8A, B). Such analyses revealed that p53-SUMO bound with stronger affinity to the promoters of several of the gene products that were found up-regulated in the micro-array platform, such as FDXR, 14-3-3-sigma, NOXA and IGF-BP3, while DNA binding was compromised on the p21, PUMA and MDM2 promoters. Significantly, p53-SUMOK37-K48R reverted the effects of p53-SUMO on several binding elements, such that it interacted less efficiently with promoters to which p53-SUMO bound more efficiently, while it enhanced binding activity on the promoters for which p53-SUMO had less affinity (FIG. 8C).

1. A method for determining the susceptibility of cells within a tissue for the induction of cell death, the method comprising determining the state of acetylation of an acetylation domain of a small ubiquitin-like modifier protein (SUMO protein) in a sample of the cells obtained from the tissue, wherein at least partial acetylation of the acetylated domain of the SUMO protein indicates that the cells within the tissue are susceptible to induction of cell death.

2. The method of claim 1, wherein the acetylation domain is fully acetylated.

3. The method of claim 1, wherein the SUMO protein is SUMO-1, SUMO-2, SUMO-3 or SUMO-4.

4. The method of claim 1, wherein determining the state of acetylation comprises the use of an antibody fragment that specifically binds to the acetylated acetylation domain of SUMO protein.

5. The method of claim 4, wherein an antibody comprises the antibody fragment.

6. The method of claim 5, wherein determining the state of acetylation comprises the use of an antibody that specifically binds to the acetylated acetylation domain of SUMO protein.

7. The method of claim 4, wherein the antibody fragment is humanized.

8. The method of claim 1, wherein the cell sample is taken from tissue or body fluid of a subject.

9. The method of claim 4, wherein determining with said antibody fragment is an in vitro assay selected from the group consisting of immunohistochemical assay, enzyme linked immunoassay (ELISA), and radioimmunoassay (RIA).

10. The method of claim 4, wherein determining with said antibody fragment is an in vivo imaging assay selected from the group consisting of X-radiography, nuclear magnetic resonance (NMR), and electron spin resonance (ESR).

11. A method for predicting the responsiveness of a subject to a cancer treatment, the method comprising determining the state of acetylation of an acetylation domain of a small ubiquitin-like modifier protein (SUMO protein) in a sample of the cells obtained from the subject in need of the cancer treatment, wherein at least partial acetylation of the acetylated domain of the SUMO protein indicates that the subject is more likely than not to respond positively to the cancer treatment.

12. The method of claim 11, wherein the acetylation domain is fully acetylated.

13. The method of claim 11, wherein the SUMO protein is SUMO-1, SUMO-2, SUMO-3 or SUMO-4.

14. The method of claim 11, wherein determining the state of acetylation comprises the use of an antibody fragment that specifically binds to the acetylated acetylation domain of SUMO protein.

15. The method of claim 11, wherein an antibody comprises the antibody fragment.

16. The method of claim 15, wherein determining the state of acetylation comprises the use of an antibody that specifically binds to the acetylated acetylation domain of SUMO protein.

17. The method of claim 14, wherein the antibody fragment is humanized.

18. The method of claim 11, wherein the cell sample is taken from tissue or body fluid of the subject

19. The method of claim 14, wherein determining with said antibody fragment is an in vitro assay selected from the group consisting of immunohistochemical assay, enzyme linked immunoassay (ELISA), and radioimmunoassay (RIA).

20. The method of claim 14, wherein determining with said antibody fragment is an in vivo imaging assay selected from the group consisting of X-radiography, nuclear magnetic resonance (NMR), and electron spin resonance (ESR).

21. The method of claim 11, wherein the cancer therapy is selected from the group consisting of podophyllotoxin, etoposide, etoposide phosphate, teniposide, cisplatin, carboplatin, oxaliplatin, mechlorethamine, cyclophosphamide, chlorambucil, ifosfamide, purines such as azathioprine and mercaptopurine, pyrimidines, Vincristine, Vinblastine, Vinorelbine, Vindesine, paclitaxel, taxol, docetaxel, irinotecan, topotecan, amsacrine, dactinomycin, doxorubicin, epirubicin and bleomycin.

22. (canceled)

23. An antibody fragment that specifically binds to an acetylation domain of a small ubiquitin-like modifier protein (SUMO protein) when the acetylation domain is at least partially acetylated.

24. The antibody fragment of claim 23, wherein the acetylation domain is fully acetylated.

25. The antibody fragment of claim 23, wherein the SUMO protein is SUMO-1, SUMO-2, SUMO-3 or SUMO-4.

26. The antibody fragment of claim 23, wherein an antibody comprises the antibody fragment.

27. The antibody fragment of claim 23, wherein the antibody fragment is humanized.

28. The antibody fragment of claim 23, wherein the antibody fragment is monoclonal.
